(19) United States
(12) Patent Application Publication
(10) Pub. No.: US 2025/0188199 A1
(43) Pub. Date: Jun. 12, 2025

(54) TERNARY DEEP EUTECTIC SOLVENT SYSTEM AND USES THEREOF

(71) Applicant: University of Connecticut, Farmington, CT (US)

(72) Inventors: Yangchao Luo, Glastonbury, CT (US); Mingyu Qiao, Storrs, CT (US); Yi Wang, Storrs, CT (US); Honglin Zhu, Farmington, CT (US)

(21) Appl. No.: 18/972,365

(22) Filed: Dec. 6, 2024

Related U.S. Application Data
(60) Provisional application No. 63/607,591, filed on Dec. 8, 2023.

(51) Int. Cl. C08B 37/08; B01D 11/02; B01D 21/26; C07K 14/78; C12N 9/00

(52) U.S. Cl. CPC C08B 37/003; B01D 11/0211; B01D 11/0288; B01D 21/262; C07K 14/78; C12N 9/00

(57) ABSTRACT

A ternary deep eutectic solvent (TDES) system for a biomolecule (for example, chitin) recovery from a biomass (for example, seafood waste), and the method of preparation of ternary deep eutectic solvent (TDES) system, and a process to differentiate/extract/recover biomolecules (such as chitin) from a biomass (such as seafood waste).

### RELATED APPLICATIONS

This application claims priority to U.S. Provisional Application No. 63/607,591 filed on Dec. 8, 2023, the contents of which are herein incorporated by reference.

### FIELD OF THE DISCLOSURE

The subject disclosure relates to compositions (for example a solvent system) and methods for extracting biomolecules from a biomass such as seafood waste. The subject disclosure particularly relates to a ternary deep eutectic solvent (TDES) system and methods for extracting high-purify chitin from seafood waste.

### BACKGROUND

Waste crustacean (lobster, crab, and shrimp) shells from seafood industry are rich sources for biomasses such as chitin, proteins, calcium salts and carotenoids. A significant share of waste shell is drained to the ocean or dumped in the landfills, leading to surface water eutrophication due to the high nitrogen content. On the other hand, this also provides an opportunity to convert landfill waste into valuable products especially if there is a sustainable approach to separate these biomasses. As the second most abundant biopolymer, chitin is produced by biological organisms and its production has reached 100 billion tons annually. It is extensively found in fungal cell walls, exoskeleton of crustacean and insects. The amount of annually discarded crustacean shells was reported to be around 6 to 8 million tons from seafood industry, which could serve as a sustainable source of chitin. Therefore, there is a need for an efficient and environmentally friendly approach to recover biomolecules such as chitin from seafood waste.

### SUMMARY OF THE INVENTION

In an aspect, disclosed is a green and efficient approach using a ternary deep eutectic solvent (TDES) system for the fractionation of high purity biomolecules such as chitin with tunable molecular weight from a biomass such as seafood waste as disclosed and described herein.

In an aspect, disclosed is a ternary deep eutectic solvent (TDES) system including a hydrogen bond acceptor (HBA) such as betaine or choline chloride (CC), a polyol-based hydrogen bond donor (HBD) such as glycerol, and an organic acid HBD such as ascorbic acid, citric acid, lactic acid, malic acid, or a combination thereof; wherein the system is used for extracting a biomolecule such as chitin from a biomass such as seafood waste.

In an aspect, disclosed is a process for treatment of biomass such as seafood waste as disclosed and described herein.

In an aspect, disclosed is a ternary deep eutectic solvent (TDES) system for recovery of a target biomolecule from a biomass, the system comprising: (a) a hydrogen bond acceptor (HBA) comprising betaine or choline chloride; (b) a first hydrogen bond donor (HBD1) comprising an organic acid; and (c) a second hydrogen bond donor (HBD2) comprising a polyol, wherein the system comprises a HBA:HBD1:HBD2 molar ratio that can be adjusted to control a molecular weight, a viscosity, and/or a morphology of the target biomolecule recovered from the biomass.

In some aspects, the organic acid is ascorbic acid, citric acid, lactic acid, malic acid, or any combination thereof.

In some aspects, wherein the polyol is ethylene glycol, glycerol, pentaerythritol, sorbitol, xylitol, or any combination thereof.

In some aspects, the HBA:HBD1:HBD2 molar ratio can be adjusted from about 1:0.05:0.1 to about 2:2:1.5 to control the molecular weight, the viscosity, and/or the morphology of the target biomolecule recovered from the biomass.

In some aspects, the HBA is betaine, the HBD1 is lactic acid, and the HBD2 is glycerol in a molar ratio of from about 1:1.9:0.1 to about 1:0.7:1.3.

In some aspects, the HBA is choline chloride, the HBD1 is lactic acid, and the HBD2 is glycerol in a molar ratio of from about 1:1.9:0.1 to about 1:0.7:1.3.

In some aspects, the target biomolecule comprises chitin, the biomass comprises seafood waste, and the molecular weight of the chitin recovered ranges from about 200 kDa to about 550 kDa without comprising purity and/or yield.

In some aspects, the HBA is betaine, the HBD1 is malic acid, and the HBD2 is glycerol in a molar ratio of from about 2:1.5:0.5 to about 2:1:1.

In some aspects, the HBA is choline chloride, the HBD1 is malic acid, and the HBD2 is glycerol in a molar ratio of from about 2:1.5:0.5 to about 2:1:1.

In some aspects, the target biomolecule comprises chitin, the biomass comprises seafood waste, and the molecular weight of the chitin recovered ranges from about 200 kDa to about 550 kDa without comprising purity and/or yield. In an embodiment, the molecular weight of the chitin recovered ranges from about 300 kDa to about 550 kDa without comprising purity and/or yield.

In some aspects, wherein the TDES system: (i) comprises lactic acid and has a viscosity of from about 15 mPa·s to about 25 mPa·s at about 80° C.; or (ii) comprises malic acid and has a viscosity of from about 80 mPa·s to about 250 mPa·s at about 80° C. In an embodiment, the TDES system: (i) comprises lactic acid and has a viscosity of from about 19 mPa·s to about 23 mPa·s at about 80° C.; or (ii) comprises malic acid and has a viscosity of from about 89 mPa·s to about 240 mPa·s at about 80° C. In an embodiment, the TDES system: (i) comprises lactic acid and has a viscosity of from about 19.23 mPa·s to about 22.28 mPa·s at about 80° C.; or (ii) comprises malic acid and has a viscosity of from about 89.10 mPa·s to about 239.69 mPa·s at about 80° C.

In an aspect, disclosed is a process for extracting a target biomolecule from a biomass, the process comprising: (a) contacting a biomass comprising a target biomolecule with a TDES system of the present disclosure under conditions effective to solubilize the target biomolecule; (b) separating the TDES containing the solubilized biomolecule from a residual biomass; and (c) recovering the target biomolecule from the TDES.

In some aspects, the biomass comprises seafood waste, seaweed, plant biomass, agricultural waste, lignocellulosic biomass, or any combination thereof. In some aspects, the target biomolecule is a protein, an enzyme, a polysaccharide, lipid or any combination thereof. In some aspects, the biomass is seafood waste comprising shrimp shells, lobster shells, crab shells, fish scales or any combination thereof, and the target biomolecule is chitin, collagen, or a combination thereof.

In some aspects, the contacting step (a) is performed at a temperature of about 50° C. to about 90° C. In some aspects, the process further comprises the step of adjusting the pH of the TDES system to precipitate the target biomolecule after separation in step (b).

In an aspect, the disclosure provides a process for treating a biomass comprising seafood waste, the process comprising: (a) magnetically stirring the biomass with a TDES of the present disclosure at a mass ratio of from about 0.5:10 to about 1.5:30 for a period of from about 1 hours to about 3 hours at a temperature ranging from about 50° C. to about 90° C. to produce an extract comprising chitin; (b) centrifuging the extract for a period ranging from about 5 minutes to about 15 minutes to produce a supernatant comprising a precipitate of the extract; and (c) drying the supernatant at a temperature ranging from about 35° C. to about 45° C. for a period of from about 16 hours to about 32 hours to produce a powder comprising chitin.

In some aspects, the process comprises cooling the extract produced in step (a) to ambient temperature prior to centrifuging the extract in step (b) and/or neutralizing the pH of the supernatant produced in step (b) prior to drying the supernatant in step (c).

In some aspects, the process comprises the step of recycling the TDES for use in subsequent extractions.

In some aspects, the process comprises optionally performing an enzymatic treatment step prior to step (a) to enhance the extraction efficiency of the TDES.

In an aspect, the disclosure provides a process for preparing a TDES system for recovery of a target biomolecule from a biomass, comprising mixing a HBA comprising betaine or choline chloride, a HBD1 comprising an organic acid; and a HBD2 comprising a polyol at a HBA:HBD1:HBD2 molar ratio selected based on a desired molecular weight, viscosity, and/or morphology of a target biomolecule to be recovered from a biomass, wherein the mixing is performed at a temperature ranging from about 75° C. to about 95° C. for a time period ranging from about 1 hour to about 3 hours until a homogenous liquid comprising the TDES system is produced.

These and other aspects of the present invention are described in more detail below.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 shows the TDES formation mechanism.

FIG. 2A and FIG. 2B show the Fourier Transform Infrared Spectroscopy (FTIR) spectra of choline chloride, carboxylic acids (i.e., lactic acid and malic acid), glycerol and synthesized deep eutectic solvents (DESs).

FIG. 3 shows the FTIR spectra of shell waste, deep eutectic solvent (DES)-extracted chitin and standard chitin.

FIG. 4 shows X-Ray diffraction (XRD) curves of shell waste, DES-extracted chitin, and standard chitin.

FIG. 5A and FIG. 5B show thermogravimetric analysis (TGA) curves of shell waste and chitins.

FIG. 6A, FIG. 6B, FIG. 6C, FIG. 6D, FIG. 6E, FIG. 6F, FIG. 6G, and FIG. 6H show scanning electron microscopy (SEM) images of snow crab shell (FIG. 6A); C-CL (chitin extracted by choline chloride and lactic acid at 1:2 ratio) (FIG. 6B); C-CLG0.5 (chitin extracted by choline chloride, lactic acid, and glycerol at 1:1.5:0.5 ratio) (FIG. 6C); C-CLG1.0 (chitin extracted by choline chloride, lactic acid, and glycerol at 1:1:1 ratio) (FIG. 6D); standard chitin (FIG. 6E); C-CM (chitin extracted by choline chloride and malic acid at 1:1 ratio) (FIG. 6F); C-CMG0.5 (chitin extracted by choline chloride, malic acid, and glycerol at 2:1.5:0.5 ratio) (FIG. 6G); C-CMG1.0 (chitin extracted by choline chloride, malic acid, and glycerol at 2:1:1 ratio) (FIG. 6H).

FIG. 7 shows the apparent viscosity at different shear rate for chitin in 5% DMAc (N,N-dimethylacetamide)/LiCl solution.

FIG. 8 shows viscosity-concentration plots of chitin fractions in 5% DMAc/LiCl solution.

FIG. 9 shows the evaluation for recycling performance of lactic acid-based DES/TDES.

FIG. 10 shows the molecular structures of five exemplary polyols of the present disclosure.

FIG. 11A and FIG. 11B shows FTIR spectra of choline chloride, lactic acid, ethylene glycol, glycerol, xylitol, and sorbitol (FIG. 11A) and CCLaEg, CCLaGly, CCLaXyl, and CCLaSor (FIG. 11B).

FIG. 12 shows flow behavior of CCLaEg, CCLaGly, CCLaXyl, and CCLaSor.

FIG. 13A, FIG. 13B, FIG. 13C, and FIG. 13D show conformations of minimization system of CCLaEg (FIG. 13A), CCLaGly (FIG. 13B), CCLaXyl (FIG. 13C), and CCLaSor (FIG. 13D).

FIG. 14 shows FTIR spectra of lobster shell, commercial chitin, and the obtained chitin.

FIG. 15 shows XRD profiles of lobster shell, commercial chitin, and the obtained chitin.

FIG. 16 shows TGA curves of lobster shell, commercial chitin, and the obtained chitin.

FIG. 17A, FIG. 17B, FIG. 17C, FIG. 17D, FIG. 17E, FIG. 17F, FIG. 17G, FIG. 17H, FIG. 17I, FIG. 17J, FIG. 17K, FIG. 17L, FIG. 17M, and FIG. 17N show SEM photographs of LS (FIG. 17A and FIG. 17B), commercial chitin (FIG. 17C and FIG. 17D), Chem-Chitin (FIG. 17E and FIG. 17F), CCLaEg-Chitin (FIG. 17G and FIG. 17H), CCLaGly-Chitin (FIG. 171 and FIG. 17J), CCLaXyl-Chitin (FIG. 17K and FIG. 17L), and CCLaSor-Chitin (FIG. 17M and FIG. 17N).

FIG. 18 shows a schematic mechanism of the DES-extracted Chitin. The figure was created using BioRender (available online).

### DETAILED DESCRIPTION

Before the disclosed processes and materials are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

As the second most abundant biopolymer, chitin is produced by biological organisms and reached 100 billion tons annually. The amount of annually discarded crustacean shells was reported to be around 6 to 8 million tons from seafood industry, which could serve as a sustainable source of chitin. However, finding a green and mild but also efficient separation of chitin from other molecules present in the shell is never an easy task due to the highly organized structure of crustacean shells. Taking a closer look at the crab shell, chitin molecules with high crystallinity are wrapped by proteins producing nanofibers with a diameter of 60 nm, and such fibers further interweave into bundles, embedded with calcium salts and carotenoids and arranged as a layered network. The development of chitin products facilitates many industries including biomedical, food, cosmetics, fertilizer and water treatment. The past decade has witnessed the increased research focus on chitin-based functional materials such as high-strength aerogel, cell carrier hydrogels, and strong single-used material.

To effectively extract chitin with high purity and yield, harsh chemicals including concentrated hydrochloride acid and sodium hydroxide are often practiced in sequence to remove minerals and proteins at industrial scale. Drawbacks come along with this practice as depolymerization and deacetylation for chitin as well as the effects of corrosive acids and caustic alkali on infrastructure and environment. The whole process is labeled as wasteful, expensive, hazardous and destructive, not to mention that tons of water is consumed for neutralization. Ionic liquids (ILs) were recently proposed for chitin recovery to overcome the disadvantages of chemical and biotechnological methods. However, recent studies disputed the greenness of ILs due to the hazardous toxicity and low degradability. As an alternative with similar properties, DESs are widely studied for their strong hydrogen bonding between hydrogen bond acceptor (typically quaternary ammonium salt) and hydrogen bond donor (usually amide, polyols, and organic acids). DESs have been gradually employed to the extraction and modification of chitin thanks to their unique characterizations such as recyclability, eco-friendliness, biocompatibility and biodegradability. Previous studies proposed two types of binary DES showing excellent extraction performance are choline chloride-lactic acid and choline chloride-malic acid for both high yield and purity. One limitation for these two DES systems are the high organic acid content of more than 50% molar ratio and strong acidic effluent for rinsing and neutralization. Furthermore, there are no investigations about the manipulation of molecular weight for extracted chitin with these two DES systems.

In an aspect, disclosed is a ternary deep eutectic solvent (TDES) system for a biomolecule (for example, chitin) recovery from a biomass (for example, seafood waste). In an aspect, the TDES system includes a hydrogen bond acceptor (HBA), a polyol-based hydrogen bond donor (HBD), and an acidic HBD. In an embodiment, the biomolecule is chitin. In an embodiment, the biomass is seafood waste, seaweeds, or a combination thereof. In an embodiment, the biomass is seafood waste. In an embodiment, the biomass is seaweeds.

In another aspect, disclosed is a ternary deep eutectic solvent (TDES) system for recovery of a target biomolecule from a biomass, the system comprising: (a) a hydrogen bond acceptor (HBA) comprising betaine or choline chloride; (b) a first hydrogen bond donor (HBD1) comprising an organic acid; and (c) a second hydrogen bond donor (HBD2) comprising a polyol, wherein the system comprises a HBA:HBD1:HBD2 molar ratio that can be adjusted to control a molecular weight, a viscosity, and/or a morphology of the target biomolecule recovered from the biomass.

The present disclosure contemplates the use of any organic acid as HBD1. Exemplary organic acids include, without limitation, ascorbic acid, citric acid, lactic acid, malic acid, or a combination thereof.

The present disclosure contemplates the use of any polyol as HBD2. Exemplary polyols include, without limitation, ethylene glycol, glycerol, pentaerythritol, sorbitol, xylitol, or a combination thereof.

Conventional caustic chitin extraction from seafood waste is unsustainable due to its significant generation of waste effluents. Disclosed herein is a green and efficient approach for the fractionation of high purity chitin with adjustable molecular weight from the biomass such as seafood waste, seaweeds, or a combination thereof. This is achieved by the ternary deep eutectic solvent (TDES) system including choline chloride (CC) as a hydrogen bond acceptor (HBA), glycerol as the polyol-based hydrogen bond donor (HBD), and lactic acid or malic acid as acidic HBD. Four TDES and two deep eutectic acids (DESs) were evaluated for their ability to recover chitin. FTIR, XRD, and TGA analysis all demonstrated excellent protein and mineral removal capabilities, as well as similar crystallinity patterns as standard chitin.

In an aspect, the weight average molecular weight, viscosity behavior and morphology of chitin extracted by DESs were varied and influenced by glycerol molar ratio. In some aspects, the HBA:HBD1:HBD2 molar ratio can be adjusted from about 1:0.05:0.1 to about 2:2:1.5 to control the molecular weight, the viscosity, and/or the morphology of the target biomolecule recovered from the biomass.

In an embodiment, the HBA is betaine, the HBD1 is lactic acid, and the HBD2 is glycerol in a molar ratio of from about 1:1.9:0.1 to about 1:0.7:1.3. In an embodiment, the HBA is choline chloride, the HBD1 is lactic acid, and the HBD2 is glycerol in a molar ratio of from about 1:1.9:0.1 to about 1:0.7:1.3. Lactic acid-based DES exhibited the controllable ability for chitin molecular weights ranging from about 200 kDa to about 600 kDa, preferably about 200 kDa to about 550 kDa, more preferably about 264 kDa to about 541 kDa without compromising purity and yield. For example, lactic acid-based DES exhibited the controllable ability for chitin molecular weights ranging from about 200 kDa to about 550 kDa, about 200 kDa to about 500 kDa, about 200 kDa to about 450 kDa, about 200 kDa to about 400 kDa, about 200 kDa to about 350 kDa, about 200 kDa to about 300 kDa, about 200 kDa to about 250 kDa, about 250 kDa to about 600 kDa, about 250 kDa to about 550 kDa, about 250 kDa to about 500 kDa, about 250 kDa to about 450 kDa, about 250 kDa to about 400 kDa, about 250 kDa to about 350 kDa, about 250 kDa to about 300 kDa, about 300 kDa to about 600 kDa, about 300 kDa to about 550 kDa, about 300 kDa to about 500 kDa, about 300 kDa to about 450 kDa, about 300 kDa to about 400 kDa, about 300 kDa to about 350 kDa, or any sub-range thereof without compromising purity and/or yield.

In an embodiment, the target biomolecule comprises chitin, the biomass comprises seafood waste, and the molecular weight of the chitin recovered ranges from about 200 kDa to about 550 kDa without comprising purity and/or yield. In some aspects, wherein the TDES system comprises lactic acid and has a viscosity of from about 15 mPa·s to about 25 mPa·s at about 80° C. In an embodiment, the TDES system comprises lactic acid and has a viscosity of from about 19 mPa·s to about 23 mPa·s at about 80° C. In an embodiment, wherein the TDES system comprises lactic acid and has a viscosity of from about 19.23 mPa·s to about 22.28 mPa·s at about 80° C. In some aspects, wherein the TDES system comprises malic acid and has a viscosity of from about 80 mPa·s to about 250 mPa·s at about 80° C. In an embodiment, the TDES system comprises malic acid and has a viscosity of from about 89 mPa·s to about 240 mPa·s at about 80° C. In an embodiment, the TDES system comprises malic acid and has a viscosity of from about 89.10 mPa·s to about 239.69 mPa·s at about 80° C.

In an embodiment, the HBA is betaine, the HBD1 is malic acid, and the HBD2 is glycerol in a molar ratio of from about 2:1.5:0.5 to about 2:1:1. In an embodiment, the HBA is choline chloride, the HBD1 is malic acid, and the HBD2 is glycerol in a molar ratio of from about 2:1.5:0.5 to about 2:1:1. In an embodiment, malic acid displayed a stronger depolymerization ability, resulting in smaller molecular weight chitin ranging from about 200 kDa to about 350 kDa or any sub-range thereof, preferably about 200 kDa to about 300 kDa, and more preferably about 204 to about 278 kDa without compromising purity and yield. This sustainable and environmentally friendly extraction system holds great potential to recover chitin from seafood waste, opening a new era for chitin further application. In an embodiment, the target biomolecule comprises chitin, the biomass comprises seafood waste, and the molecular weight of the chitin recovered ranges from about 200 kDa to about 300 kDa without comprising purity and/or yield.

In an aspect, disclosed is a ternary DES system for a biomolecule recovery. In an embodiment, the ternary DES system includes glycerol-added betaine-lactic acid (LA) or CC-malic acid (MA). In an aspect, disclosed is a ternary DES system for a biomolecule recovery. In an embodiment, the ternary DES system includes glycerol-added choline chloride (CC)-lactic acid (LA) or CC-malic acid (MA). The ternary DES system is designed to achieve the desirable outcome for biomolecules such as chitin recovery. In an embodiment, the ternary DES system for a biomolecule recovery includes glycerol, betaine, and LA. In an embodiment, the ternary DES system for a biomolecule recovery includes glycerol, CC, and LA. In an embodiment, the ternary DES system for a biomolecule recovery includes glycerol, betaine, and MA. In an embodiment, the ternary DES system for a biomolecule recovery includes glycerol, CC, and MA. In an embodiment, the biomolecule recovered is chitin. In an embodiment, the biomolecule is recovered from a biomass. In an embodiment, the biomolecule is chitin. In an embodiment, the biomass is seafood waste. In an embodiment, the molecular weight of recovered chitin ranges from about 200 kDa to about 600 kDa or any sub-range thereof. In an embodiment, the molecular weights of recovered chitin ranges from about 200 kDa to about 550 kDa or any sub-range thereof. In an embodiment, the molecular weights of recovered chitin ranges from about 264 kDa to about 541 kDa. In an embodiment, the molecular weight of recovered chitin ranges from about 204 to about 278 kDa.

In an embodiment, the ternary DES system includes betaine (B), lactic acid (LA) and glycerol (Gly) (i.e., B:LA:Gly) at molar ratios of about 1:1.9:0.1 to about 1:0.7:1.3 or any sub-range thereof, preferably about 1:1.5:0.5 to about 1:1:1. In an embodiment, the ternary DES system includes B:LA:Gly at molar ratios of about 1:1.5:0.5. In an embodiment, the ternary DES system includes B:LA:Gly at molar ratios of 1:1:1. In an embodiment, the ternary DES system includes betaine (B), malic acid (MA) and glycerol (Gly) (i.e., B:MA:Gly) at molar ratios of about 2:1.5:0.5 to about 2:1:1 or any sub-range thereof. In an embodiment, the ternary DES system includes B:MA:Gly at molar ratios of about 2:1.5:0.5. In an embodiment, the ternary DES system includes B:MA:Gly at molar ratios of about 2:1:1. In an embodiment, the ternary DES system includes a HBA:HBD ratio from about 1:1 to about 1:2 or any sub-range thereof. In an embodiment, the TDES system includes betaine (B), lactic acid (LA) and ethylene glycol at a molar ratio of from about 0.5:0:5:0.5 to about 2:2:2. In an embodiment, the TDES system includes betaine (B), lactic acid (LA) and ethylene glycol at a molar ratio of about 1:1:1. In an embodiment, the TDES system includes betaine (B), lactic acid (LA and pentaerythritol at a molar ratio of from about 0.5:0:5:0.5 to about 2:2:2. In an embodiment, the TDES system includes betaine (B), lactic acid (LA) and pentaerythritol at a molar ratio of about 1:1:1. In an embodiment, the TDES system includes betaine (B), lactic acid (LA and xylitol at a molar ratio of from about 0.5:0:5:0.5 to about 2:2:2. In an embodiment, the TDES system includes betaine (B), lactic acid (LA) and xylitol at a molar ratio of about 1:1:1. In an embodiment, the TDES system includes betaine (B), lactic acid (LA and sorbitol at a molar ratio of from about 0.5:0:5:0.5 to about 2:2:2. In an embodiment, the TDES system includes betaine (B), lactic acid (LA) and sorbitol at a molar ratio of about 1:1:1.

In an embodiment, the ternary DES system includes choline chloride (CC), lactic acid (LA) and glycerol (Gly) (i.e., CC:LA:Gly) at molar ratios of about 1:1.9:0.1 to about 1:0.7:1.3 or any sub-range thereof, preferably about 1:1.5:0.5 to about 1:1:1. In an embodiment, the ternary DES system includes CC:LA:Gly at molar ratios of about 1:1.5:0.5. In an embodiment, the ternary DES system includes CC:LA:Gly at molar ratios of 1:1:1. In an embodiment, the ternary DES system includes choline chloride (CC), malic acid (MA) and glycerol (Gly) (i.e., CC:MA:Gly) at molar ratios of about 2:1.5:0.5 to about 2:1:1 or any sub-range thereof. In an embodiment, the ternary DES system includes CC:MA:Gly at molar ratios of about 2:1.5:0.5. In an embodiment, the ternary DES system includes CC:MA:Gly at molar ratios of about 2:1:1. In an embodiment, the ternary DES system includes a HBA:HBD ratio from about 1:1 to about 1:2 or any sub-range thereof. In an embodiment, the TDES system includes choline chloride (CC), lactic acid (LA) and ethylene glycol at a molar ratio of from about 0.5:0:5:0.5 to about 2:2:2. In an embodiment, the TDES system includes choline chloride (CC), lactic acid (LA) and ethylene glycol at a molar ratio of about 1:1:1. In an embodiment, the TDES system includes choline chloride (CC), lactic acid (LA and pentaerythritol at a molar ratio of from about 0.5:0:5:0.5 to about 2:2:2. In an embodiment, the TDES system includes choline chloride (CC), lactic acid (LA) and pentaerythritol at a molar ratio of about 1:1:1. In an embodiment, the TDES system includes choline chloride (CC), lactic acid (LA and xylitol at a molar ratio of from about 0.5:0:5:0.5 to about 2:2:2. In an embodiment, the TDES system includes choline chloride (CC), lactic acid (LA) and xylitol at a molar ratio of about 1:1:1. In an embodiment, the TDES system includes choline chloride (CC), lactic acid (LA and sorbitol at a molar ratio of from about 0.5:0:5:0.5 to about 2:2:2. In an embodiment, the TDES system includes choline chloride (CC), lactic acid (LA) and sorbitol at a molar ratio of about 1:1:1.

Glycerol was introduced for chitin recovery in 2018 after the initial successful practice of hot glycerol followed by citric acid treatment. Glycerol as a nontoxic, biocompatible, and inexpensive byproduct from biodiesel industry, is also well known for its high boiling and nonvolatile properties. Zhang's study investigated the extraction performance of the CC-glycerol DES with small amount of acetic acid but a high temperature at 120° C. was needed (Zhang et al., 2022, Facile production of chitin from shrimp shells using a deep eutectic solvent and acetic acid. RSC Advances, 12 (35), 22631-22638). Inventors unexpectedly discovered a milder and eco-friendly route for chitin recovery with less amount of organic acid to reduce the possible hydrolysis of chitin in traditional binary DES. Additionally, glycerol was included into DESs for protecting chitin chains from acid hydrolysis under organic acid conditions. The extraction performance of different DES indicating yield, molecular weight, and purity was systematically compared. Thermal properties, morphology, and rheological behavior of chitins were characterized to give the complete profile.

In an aspect, disclosed is a method to prepare a ternary deep eutectic solvent (TDES) system disclosed herein. The method includes providing a hydrogen bond acceptor (HBA) such as choline chloride; providing and mixing a polyol-based hydrogen bond donor (HBD) such as glycerol; and providing and mixing an acidic HBD such as lactic acid or malic acid.

In an aspect, disclosed is a process for preparing a TDES system for recovery of a target biomolecule from a biomass, comprising mixing a HBA comprising betaine or choline chloride, a HBD1 comprising an organic acid; and a HBD2 comprising a polyol at a HBA:HBD1:HBD2 molar ratio selected based on a desired molecular weight, viscosity, and/or morphology of a target biomolecule to be recovered from a biomass, wherein the mixing is performed at a temperature ranging from about 75° C. to about 95° C. for a time period ranging from about 1 hour to about 3 hours until a homogenous liquid comprising the TDES system is produced.

In an aspect, disclosed is a process to differentiate/extract/recover biomolecules (such as chitin) from a biomass (such as seafood waste). In some aspects, the process includes treating a biomass such as seafood waste with a TDES system disclosed herein to provide a mixture; subjecting the mixture to a heating process; and separating a precipitate to afford a biomolecule. In an embodiment, the biomass is seafood waste, seaweeds, or a combination thereof. In an embodiment, the process further includes centrifuging the mixture after the heating process. In an embodiment, the heating process is performed at about 80° C. In an embodiment, the heating process is performed at about 80° C. for about 2 hours. In an embodiment, the process further includes washing/rinsing the precipitate with deionized (DI) water several times until the pH of the supernatant becomes neutral. In an embodiment, the process further includes drying the precipitate at about 40° C. In an embodiment, the drying of the precipitate is performed at about 40° C. for about 24 hours. In an embodiment, the biomolecule is chitin.

In an aspect, disclosed is a process for extracting a target biomolecule from a biomass, the process comprising: (a) contacting a biomass comprising a target biomolecule with a TDES system of the present disclosure under conditions effective to solubilize the target biomolecule; (b) separating the TDES containing the solubilized biomolecule from a residual biomass; and (c) recovering the target biomolecule from the TDES.

The TDES systems of the present disclosure can be used to extract target biomolecules from a variety of different types of biomass. In some aspects, the biomass is seafood waste, seaweed, plant biomass, agricultural waste, lignocellulosic biomass, or any combination thereof. In other aspects, the biomass is seafood waste. In an embodiment, the seafood waste comprises shrimp shells. In an embodiment, the seafood waste comprises lobster shells. In an embodiment, the seafood waste comprises crab shells. In an embodiment, the seafood waste comprises fish scales. In some embodiments, the seafood waste comprises any combination of shrimp shells, lobster shells, crab shells, and/or fish scales.

In some aspects, the seafood waste comprises shrimp shells, lobster shells, crab shells, and/or fish scales and the target biomolecule is chitin, collagen or a combination thereof.

The TDES systems of the present disclosure can be used to extract any desirable target biomolecule. In some aspects, the target biomolecule is a protein, an enzyme, a polysaccharide, lipid or any combination thereof.

The skilled artisan will ready appreciate which conditions are effective to solubilize a target biomolecule using a TDES system of the present disclosure, including for example temperature. In some aspects, contacting step (a) is performed at a temperature of about 50° C. to about 90° C. In other aspects, the process further comprises a step of adjusting the pH of the TDES system to precipitate the target biomolecule after separation in step (b).

In an aspects, disclosed is a process for treating a biomass comprising seafood waste, the process comprising: (a) magnetically stirring the biomass with a TDES of the present disclosure at a mass ratio of from about 0.5:10 to about 1.5:30 for a period of from about 1 hours to about 3 hours at a temperature ranging from about 50° C. to about 90° C. to produce an extract comprising chitin; (b) centrifuging the extract for a period ranging from about 5 minutes to about 15 minutes to produce a supernatant comprising a precipitate of the extract; and (c) drying the supernatant at a temperature ranging from about 35° C. to about 45° C. for a period of from about 16 hours to about 32 hours to produce a powder comprising chitin. In some embodiment, the step (a) of the process is performed at a temperature ranging from about 70° C. to about 90° C. to produce an extract comprising chitin.

In some aspects, the process comprises cooling the extract produced in step (a) to ambient temperature prior to centrifuging the extract in step (b) and/or neutralizing the pH of the supernatant produced in step (b) prior to drying the supernatant in step (c). In some aspects, the process comprises recycling the TDES for use in subsequent extractions. In other aspects, the process further comprises optionally performing an enzymatic treatment step prior to step (a) to enhance the extraction efficiency of the TDES.

The disclosed solvent system and process provides the following advantages over the currently used systems and processes: all solvents are environmentally friendly; ingredients used in the formulation are relatively low-cost; extraction time is short (about 2 hours), compared to several days in conventional extraction method; high purity and yield of final products are attainable; molecular weight of extracted biomolecules is tunable; TDES showed excellent chitin extraction ability even after three recycles.

The present disclosure is illustrated and further described in more detail with reference to the following non-limiting examples. Section headings as used in this section and the entire disclosure herein are merely for organizational purposes and are not intended to be limiting.

### EXAMPLES

### Example 1

### Methods and Materials

### Materials

Snow crab (Chionoecetes opilio) legs and claws were collected from the grocery store Big Y near the University of Connecticut (Storrs, CT, USA). The crab was once boiled for 5 min and then peeled to remove all the meat and any impurities to collect crab shell. The shells were then dried in the oven at 60° C. for 12 hours and then grinded by a kitchen grinder with a maximum powder size at around 200 μm. Standard α-chitin samples were purchased by Thermo Fisher Scientific. Glycerol (99+%) and N, N-Dimethylacetamide (DMAc) were purchased from Acros-Organics. All other chemicals were analytical grade and purchased from Thermo Fisher Scientific unless stated otherwise.

### Synthesis of DES

TDESs were prepared by mixing choline chloride (CC), lactic acid (LA) and glycerol (Gly) at two molar ratios, i.e., CC:LA:Gly of 1:1.5:0.5 and 1:1:1. DES were prepared by mixing the CC and LA in the molar ratio 1:2. The preparation temperature was set as 80° C. and the mixture was under the magnetic stirring until a clear and homogenous liquid was acquired. For the formula with malic acid (MA), the preparation was following the similar procedures as LA with minor modification. Choline chloride, malic acid and glycerol were mixed at different molar ratios and synthesis temperature as shown in Table 1.

TABLE 1
Synthesis conditions for TDES

| | Sample | | | | | | Viscosity |
|---|---|---|---|---|---|---|---|
| | code for | | | Molar | Synthesis | | at 80° C. |
| DES system | chitin | HBA | HBD | ratio | temperature | Color | (mPa · s) |
| CCLA | C-CL | Choline | Lactic | 1:2 | 80° C. | Colorless | 21.67 |
| | | chloride | acid | | | | |
| CCLAGly0.5 | C-CLG0.5 | | Lactic | 1:1.5:0.5 | 80° C. | Colorless | 19.23 |
| | | | acid + | | | | |
| | | | glycerol | | | | |
| CCLAGly1.0 | C-CLG1.0 | | Lactic | 1:1:1 | 80° C. | Colorless | 22.28 |
| | | | acid + | | | | |
| | | | glycerol | | | | |
| CCMA | C-CM | | Malic | 1:1 | 90° C. | Light | 89.10 |
| | | | acid | | | amber | |
| CCMAGly0.5 | C-CMG0.5 | | Malic | 2:1.5:0.5 | 90° C. | Amber | 239.69 |
| | | | acid + | | | | |
| | | | glycerol | | | | |
| CCMAGly1.0 | C-CMG1.0 | | Malic | 2:1:1 | 90° C. | Amber | 137.91 |
| | | | acid + | | | | |
| | | | glycerol | | | | |

### Characterization of TDES

The Fourier transform infrared (FTIR) spectroscopy was carried out using a Nicolet IS5 spectrometer (Thermo Scientific, Waltham, MA, USA). IR spectra for the chitin samples were measured with a range of 500-4000 cm⁻¹ at a resolution of 4 cm⁻¹ and data analysis was performed on OMNIC software. The apparent viscosity of DES was measured by the Rheometer (Anton Paar, MCR 302) at 80° C.

### Chitin Recovery

The crab shell and DES solutions were mixed together at a mass ratio of 1:20 and heated at 80° C. for 2 h under magnetic stirring. After heating process, the obtained extract was cooled down to room temperature by adding an equal volume of DI water. The mixture was then centrifuged (6200×g) for 10 min and the precipitate was collected and then was rinsed with DI water several times until the pH of the supernatant became neutral. Chitin powder was obtained by oven drying at 40° C. for 24 h and kept in a desiccator for future use. The code for different samples were set as “C-” (stands for Chitin), followed by an abbreviation of different DES systems as shown in Table 1.

### Recycling of TDES

After the removal of precipitate, the supernatant composed of DES and water was collected. Water was removed by rotary evaporation. DES was then reused for chitin extraction following the same procedures as described in Paragraph herein.

### Chemical Composition Analysis

The chemical composition analyses were conducted to measure the content of residual ash, protein and moisture. The ash content was determined gravimetrically by TGA (TA Instruments Q500-0188, USA). Around 0.015 g sample was heated at a rate of 10° C./min until 600° C. at air atmosphere and held at 600° C. for 30 min. The measurement of protein content followed a previously reported Lowery method. Briefly, around 0.5 g DES-isolated chitins were pre-treated with 10 mL 5% NaOH and heated to 95° C. for 2.5 h. After that, the mixture was centrifuged at 2700×g for 5 min to collect supernatant for protein measurement. Bovine serum albumin (BSA) was used as the model protein solution to provide a standard concentration-absorbance curve at 562 nm wavelength. Moisture content was measured by heating samples to 200° C. until a constant weight by Q500 TGA instrument. Yield and purity were respectively calculated as equation (1) and (2)

$$Yield = {W}_{1} / {W}_{0} \qquad ( 1 )$$

$$Purity = ( {W}_{1} - {W}_{pro} - {W}_{mineral} ) / {W}_{1} \qquad ( 2 )$$

Yield was calculated as the ratio of the extracted chitin weight (W₁) and initial shell weight (W₀). Wₚᵣₒ and Wₘᵢₙₑᵣₐₗ refer to the weight of protein and mineral in extracted chitins. Purity was calculated as the ratio of pure chitin and extracted chitin weight (W₁), where pure chitin was the result of extracted chitin weight (W₁) subtracting the weight of proteins (Wₚᵣₒ) and minerals (Wₘᵢₙₑᵣₐₗ).

### Characterization of Isolated Chitin

The FTIR spectroscopy of DES extracted chitin was performed same as [0038]. X-ray diffraction (XRD) of the chitins was observed by a Bruker D2 Phaser Diffractometer (Brüker, Germany). XRD Records were performed in the 2θ range of 5° 40° with a step size of 0.01° and 5 s/step as the counting time. The crystalline index (CrI: %) was measured as the following equation:

$${CrI}_{110} = \vert \frac{( {I}_{110} - {I}_{a m} )}{{I}_{110}} \vert \times 100 \qquad ( 3 )$$

where I₁₁₀ is the maximum intensity at 2θ≅19° and Iₐₘ is the intensity of amorphous diffraction at 2θ≅12.6°. The thermal gravimetric analysis (TGA) curve was carried out on Q500 (TA Instruments Q500-0188, USA) under a nitrogen atmosphere. In detail, 0.015 g of the sample was excited from 25° C. to 800° C. at a rate of 10° C./min with a nitrogen gas flow of 40.0 mL/min. To obtain the viscosity curve, chitin samples were dissolved into 5% LiCl/DMAc solution at a concentration of 0.05 g/dL. To fully dissolve chitin, the mixture was heated to 80° C. for 2 h and the solution was centrifuged at 2250×g for 6 min to remove any impurities before further measurement. The viscosity flow curve of chitin solutions was performed on a Rheometer (MCR 302, Anton Paar) through a concentric cone plate (diameter: 50 mm, angle: 1°). The procedure was set as steady shear experiments, and the shear range was set from 0.01 s^{-1 1000} s⁻¹ at 30° C. The cone plate gap was chosen as 0.098 mm for all measurements. The field emission scanning electron microscopy (FE-SEM, JSM-6330F, JEOL Ltd., Tokyo, Japan) was employed to analyze the surface morphology of snow crab shell and isolated chitins. Before observation, the samples were coated with a Pb—Au layer under vacuum using a sputter coater for conductivity.

### Molecular Weight Measurement

The viscosity-based Mw measurement was performed by Ubbelohde capillary viscosity (Φ0.4-0.5 mm) at 25° C. Before the test, chitin samples were dissolved into 5% (w/w) DMAc/LiCl solvent at least four different concentrations from 0.03 g/dL to 0.09 g/dL. The solutions were centrifugated at 2100×g for 5 min to remove insoluble particles. Specific viscosity (ηₛₚ) of DES-extracted chitin was calculated by this equation

$${η}_{sp} = ( t - {t}_{0} ) / {t}_{0} \qquad ( 4 )$$

where t and to are the times for chitin solutions and DMAc/LiCl solvent flowing out of the capillary, respectively. Intrinsic viscosity ([η]) is calculated by extrapolating the linear regression of specific viscosity and concentration to y-intercept with the M_{w} ranging from 80 kDa to 710 kDa. Weight average molecular weight (M_{w}) is related to intrinsic viscosity by Mark-Houwink-Sakurada (MHS) equation:

$$[ η ] = 7.6 * {10}^{- 5} {M}_{w}^{0.95} \qquad ( 5 )$$

### Result and Discussion

### DES Formation

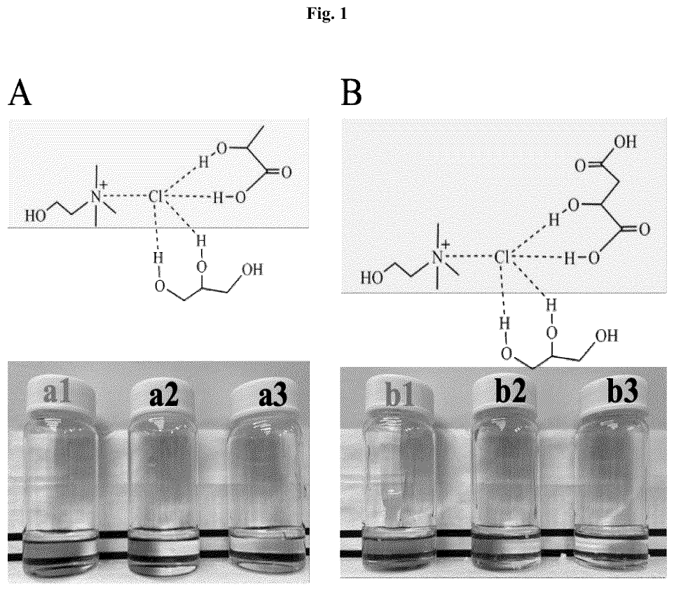

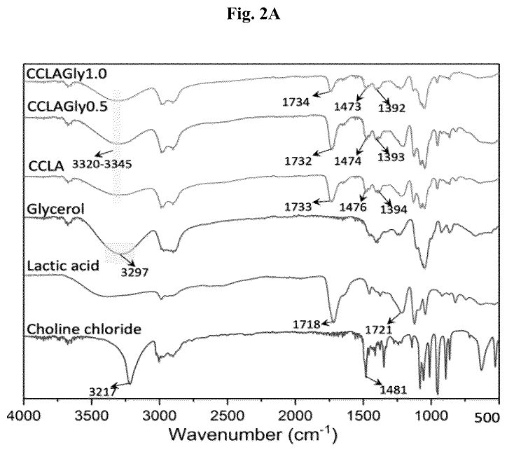

DES and TDES were successfully prepared as they exhibited a clear and homogeneous liquid state under room temperature (FIG. 1, vials a1 to b3). Choline chloride functions as HBA and can form multiple hydrogen bonds with hydroxyl and carboxyl groups on glycerol and organic acid. This hydrogen bonding network greatly decreased the melting point of the system thus showing a stable liquid state. Malic acid based DES indicated a more viscous property than lactic acid-based DES at 80° C. This difference was attributed to malic acid having more carboxyl groups than lactic acid, thus forming more hydrogen bonds. These DES systems rich in hydrogen bonds allow the separation of chitin from other compounds by breaking the original hydrogen bonding in crab shells.

### Characterization of DES

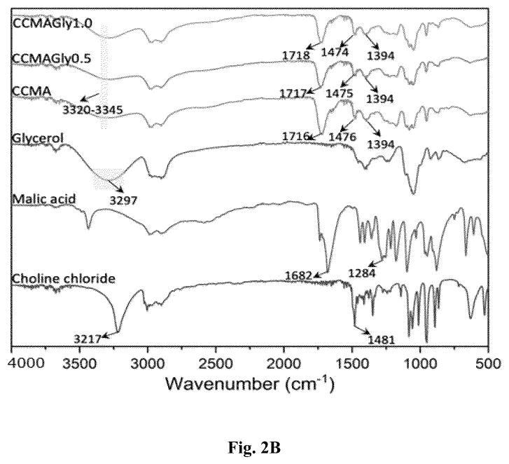

The FTIR has been widely applied to analyze the formation and strength of hydrogen bonds in DESs between HBD and HBA. The IR spectra of choline chloride, organic acid, glycerol and DESs are shown in FIG. 2. The differences of vibration bands and bandwidths in different DES systems were observed. Choline chloride, as the quaternary ammonium salt, peaks at around 1477 cm⁻¹ are related to the scissor angular deformation of the —CH₂ groups, which overlap with the peaks of the —CH₃ groups. These characteristic peaks were also observed in the spectrum of both binary and ternary DES systems. Another band that maintains in the spectrum of the binary and ternary mixtures was the peak around 1709 cm⁻¹, which relates to the stretching of —C—O bonds in the carboxylic acids (lactic acid and malic acid). The formation of hydrogen bonds between choline chloride and carboxylic acids in binary mixtures are directly evidenced by the bands at 1290 cm⁻¹ and 1390 cm⁻¹, which corresponds to characteristic of carboxylic acid dimers, C—O bond stretching and C—O—H group. However, significant reduction in their intensity was noted, revealing that the carboxylic acid dimers were broken during the DES synthesis. The addition of glycerol was shown to be part of HBD because the FTIR absorption bands of the stretching vibration of O—H groups (3320-3345 cm⁻¹) of glycerol as HBD shifted to lower wavenumber in ternary DES systems, thus proving that the O—H functional group of HBD takes part in the formation of the hydrogen bonding among anions of choline chloride.

### Characterization of Chitin

### Chemical Analysis

In order to obtain the pure chitin with high molecular weight for further use, the chemical composition of DES-extracted chitin was analyzed (Table 2). The decolorization step is unnecessary due to the shallow color of snow crab shell. Six samples all showed 4%-5% moisture content which was about 1% higher than other binary-DES extracted chitin. The compositional analyses for the original snow crab shell were protein (17.65±0.76%) and mineral (50.89±1.03%), which were in accordance with the previous study. After the DES treatment, the ash residue in all the samples decreased significantly. For the effect of glycerol, an increased ash content was found with an increasing molar ratio of glycerol. This was explained by the decreased amount of malic acid and the less acidic environment. After DES treatment, the protein content was dramatically reduced to only 1.63-2.54% suggesting the desirable deproteinization ability of both glycerol-added ternary DES and binary DES. Protein that remained in chitin samples is known to work as a binder and enhancer for chitin nanofiber production, resulting in greater diameter of obtained chitin nanofiber. Choline chloride-based DES showed strong protein dissolution and extraction ability especially when the HBDs are dihydric alcohols. The yields of all six chitin samples did not show any appreciable differences. A slightly higher yield of 36.75% was observed at C-CLG0.5, which could be explained by the low viscosity of CLG0.5 at 80° C. Excessive viscosity has been shown to hinder the penetration of DES to chitin as well as the mixing and separation process. It is interesting to know the addition of glycerol into binary DES did not hamper the extraction ability of the organic acid-based DES. Previous studies showed that DES composed of choline chloride and glycerol at the 1:2 molar ratio isolated chitin simultaneously with minerals. When TDES synthesized with organic acid, in this system, it was demonstrated that the purity of chitin increased significantly. Additionally, the TDES solvent systems achieve controllable molecular weight of chitin.

TABLE 2
Chemical composition of crab shells and recovered chitin samples

| | | Protein | | Ash | | |
|---|---|---|---|---|---|---|
| Sample | | content | Moisture | content | | Crystallinity |
| code | Yield (%) | (%) | (%) | (%) | Purity (%) | (%) |
| C-CL | 33.97 ± 4.91 | 1.63 ± 0.58 | 5.72 ± 0.30 | 1.74 ± 0.11 | 96.63 ± 0.69 | 82.46 |
| C-CLG0.5 | 36.75 ± 0.51 | 2.00 ± 0.21 | 4.145 ± 0.05 | 1.47 ± 0.03 | 96.53 ± 0.24 | 83.62 |
| C-CLG1.0 | 34.27 ± 4.04 | 2.31 ± 1.24 | 4.325 ± 0.02 | 1.14 ± 0.06 | 96.55 ± 1.19 | 82.51 |
| C-CM | 33.95 ± 0.88 | 2.36 ± 0.88 | 4.01 ± 0.15 | 1.17 ± 0.08 | 96.48 ± 0.81 | 85.14 |
| C-CMG0.5 | 32.15 ± 0.75 | 1.77 ± 0.13 | 4.25 ± 0.20 | 1.72 ± 0.15 | 96.52 ± 0.02 | 87.27 |
| C-CMG1.0 | 34.38 ± 0.10 | 2.54 ± 1.52 | 5.45 ± 0.35 | 1.78 ± 0.19 | 95.69 ± 1.33 | 84.79 |

### FTIR

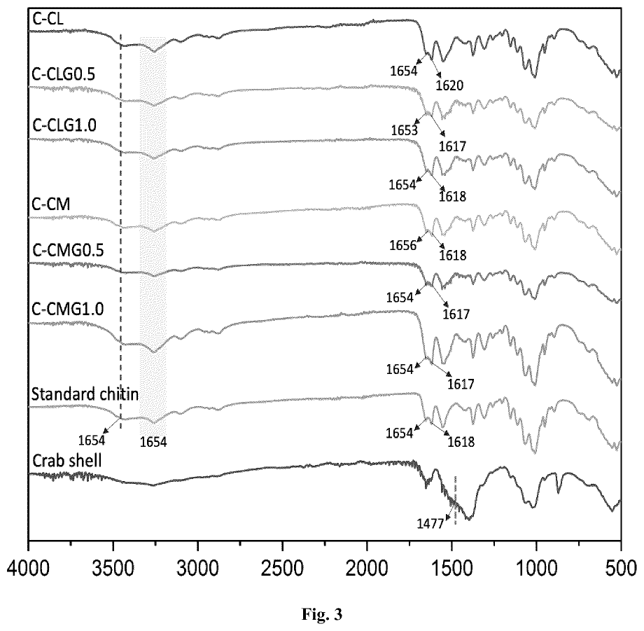

Alpha-chitin is the major component for shell chitin, which is an antiparallel linear structure with abundant inter and intra hydrogen bonds leading to high crystallinity. Intermolecular hydrogen bonds (NH . . . C═O and C6—OH . . . O—C) and intramolecular hydrogen bonds (C3—OH . . . C5) are major contributions to the high crystallinity stabilizing the chitin polymer. The FTIR spectra of snow crab shell, standard chitin (commercial product) and as-prepared chitins (DES-extracted) are shown in FIG. 3. The symmetric stretching vibrations of —NH₂ and —OH were reflected by the peaks at 3471 cm⁻¹ and 3256 cm⁻¹. The intensity of hydrogen bond was reflected by the width of these two peaks. The C—H stretching with an absorbance peak at 2875 cm⁻¹ was also observed, which was one of the characteristic peaks of chitin. The amide I band usually falls into 1600-1700 cm⁻¹ but showed a split at 1656 cm⁻¹ and 1621 cm⁻¹, which was due to the existence of the hydrogen bond both intermolecular and intramolecular. Amide II and amide III at 1552 cm⁻¹ and 1311 cm⁻¹ respectively were also noted on the spectra. Compared with standard chitin, the snow crab shell showed a quite different spectrum, which can be explained by the proteins existing in the matrix. The amide band at 1477 cm⁻¹ was a characteristic peak of protein molecules, which was absent in the DES-extracted chitins. Among the obtained samples, C-CL showed more similarity with standard chitin and clearer spectra patterns than chitins from ternary DES with the addition of glycerol. For chitin from the malic acid-based DES, the addition of glycerol did not show an obvious difference from the C-CM. This could be explained by the stronger acidic conditions in the malic-based DES systems which weakened the effect of glycerol.

### XRD

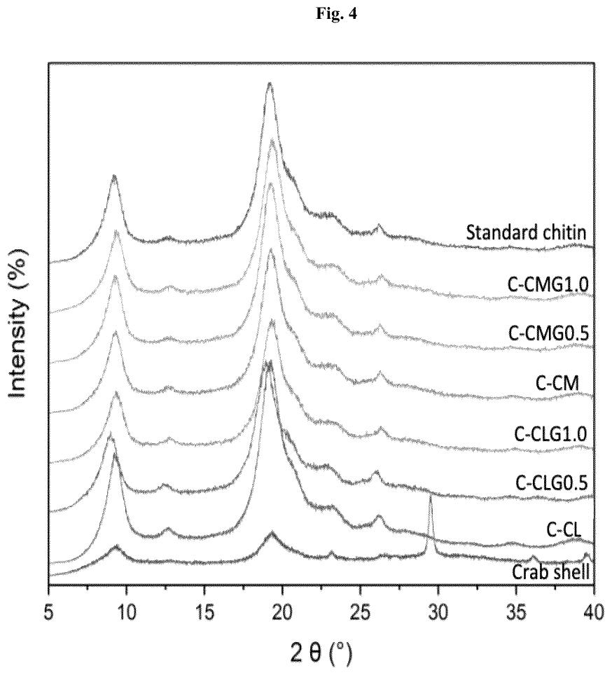

XRD patterns of the standard, DES-extracted chitin and shell waste are presented in FIG. 4. The two most obvious crystalline reflections were located at 9.2° and 19.2° for both standard chitin and DES-extracted chitin. According to crystalline index equation, DES-extracted chitin all had a slightly higher crystalline index than traditional chemical extracted chitin (81.62%). Except for these two peaks, another two pale diffraction peaks at 12.64° and 23.40° were also identified from the result. The intensity and the respective 20 degree values suggested the stable structure of α-chitin. Purity can also be obtained from the XRD results. According to FIG. 4 DES-extracted chitins were free from the crystalline peaks of CaCO₃, which was the peak at around 30° and was observed for shell waste diffraction patterns. These results confirmed the removal of protein from shell waste with previous studies.

### TGA

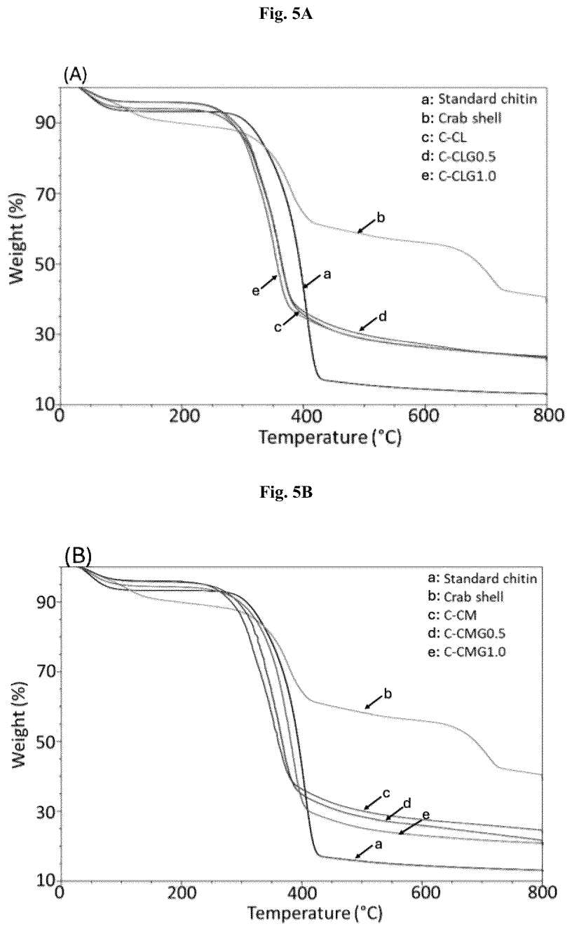

FIG. 5 presents the thermogravimetric analysis (TGA) under nitrogen environment. TGA plot could be divided into three regions regarding to the thermal degradation rate at different temperature ranges. In detail, first mass loss between room temperature and 110° C. for all samples could be explained by the evaporation of chemisorbed water. The second decomposition stage occurring between 250° C. and 400° C. were noticeable for all the samples where the polymer structure of chitin broke into oligosaccharides and finally thermally degraded. The decomposition rate at the second stage varied, which was mainly explained by the different molecular weights and crystallinity. Usually, polymer with a lower molecular weight usually has a lower thermal stability than the one with a higher molecular weight. For malic acid based TDES, an increasing decomposition temperature was found for C-CMG1.0, which could be explained by the high molecular weight of chitin due to the less acidic extraction environment. The last degradation stage between 650° C. and 750° C. was only observed for crab shell which was caused by the decomposition of calcium carbonate into calcium oxide and carbon dioxide. This stage was absent from DES-extracted chitin which was in accordance with the low ash residue.

### Surface Morphology

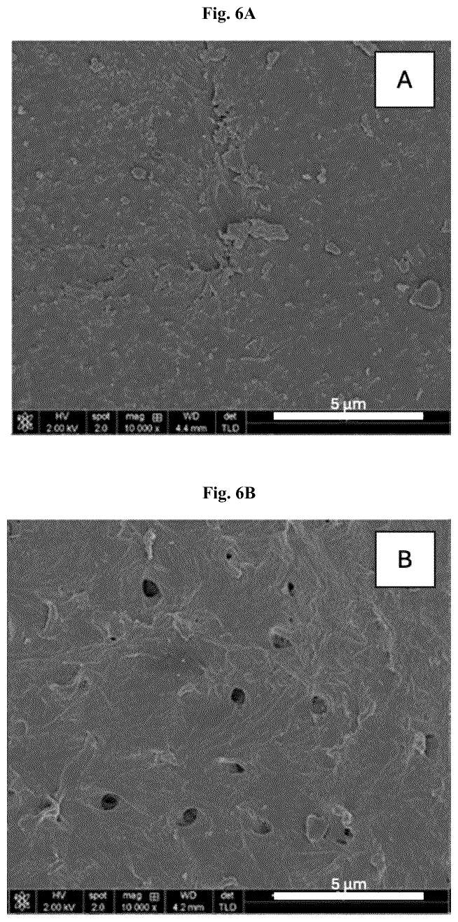

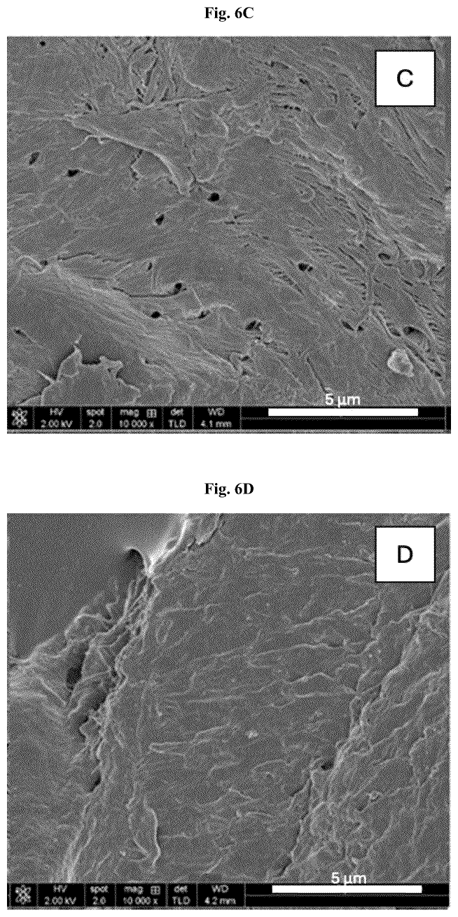

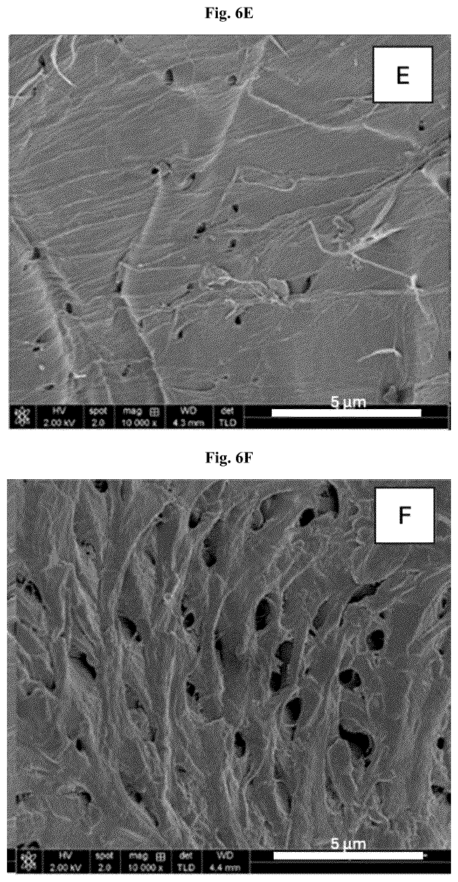

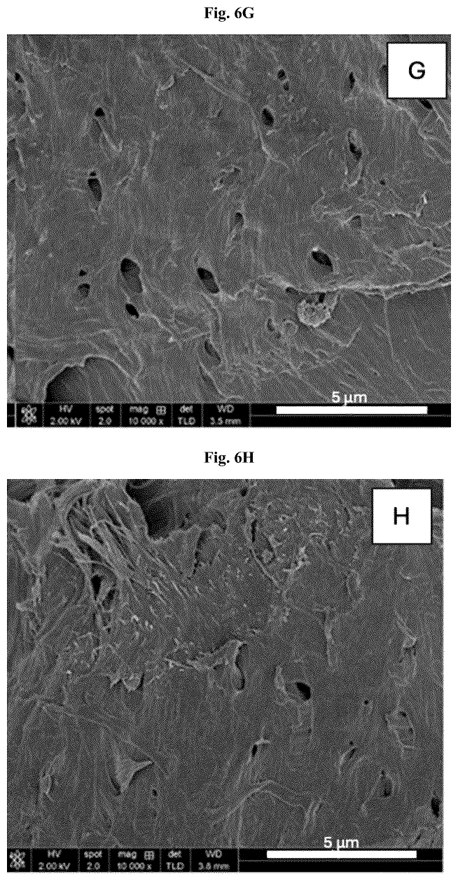

Crab shells and extracted chitins were examined by SEM to analyze their surface morphology with a scale bar set at 5 μm (FIG. 6). Significant differences could be observed between extracted chitins and crab shells. The SEM image of crab shell revealed a rigid and rough surface with aggregates due to the presence of proteins and minerals (FIG. 6a). But the Bouligand structure formed by the interweaving and periodically branching chitin-protein fibers could not be seen. Instead, the mineralized fibrous chitin-based networks were exposed in all extracted chitins (FIG. 6b-FIG. 6h). It is interesting to observe the fibrous structure with high aspect ratio on the surface by acid-alkali treatment. No protein or mineral aggregates were observed, which was in agreement with FTIR and XRD data, evidencing the elimination ability of DES. Moreover, C-CM and C-CMG0.5 exhibited larger pore size and porosity on the surface compared to other groups, which could be attributed to the harsh acid conditions and strong penetration ability of malic acid induced by consistent heating at 80° C. Previous studies also showed a similar corroded surface and hydrolysis of chitin by choline chloride and malic acid. Thick bundle of chitin fibers were arranged side by side with width ranging from 200 nm to 500 nm (FIG. 6f and FIG. 6c). The porous structure was favored by prospective applications such as drug delivery and release and adsorption of metal ions and dyes.

### Steady Shear Properties

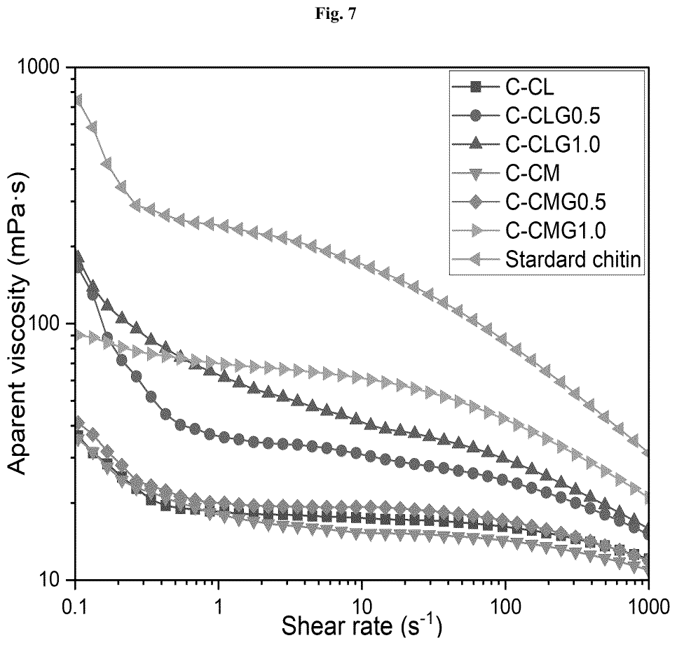

Apparent viscosity under steady state shear was used to reflect the polymer average molecular weight and degree of polymerization (FIG. 7). All chitin samples exhibited a clear shear thinning region from 0.1 s⁻¹ to 1000 s⁻¹ and clear infinite-shear Newtonian Plateau above 10 s⁻¹. Similar shear thinning patterns were observed from glycerol-hydrochloride acid extracted chitin solutions from previous study. Chitin molecules entangled with neighboring molecules in the start-up state. With the increase in shear rate, chitin backbone was gradually stretched and eventually the whole structure collapsed as chitin became aligned due to the high shear rate. Their flow behaviors fit cross model well except that the limited start-up shear was not low enough to allow the existence of zero-shear Newtonian Plateau. Among them, standard chitin showed the highest viscosity than any other samples for the whole shear rate. This is in accordance with SEM results where standard chitin showed the smaller pore size and lower pore density. For DES-extracted chitin, C-CLG1.0 and C-CMG1.0 showed higher viscosity than other chitin samples which were related to their relatively intact morphology and less acidic environment. C-CL, C-CM and C-CMG0.5 were found to show a similar shear thinning behavior. For shear rate under 0.1 to 1 s⁻¹, chitin chains slowly oriented along the shear direction, causing a slowly decreasing viscosity. The plateau under 1 to 100 s⁻¹ was due to the increased molecule entanglement level which was disrupted at high shear rate around 100 s⁻¹ and a gradual viscosity decrease was observed.

### Molecular Weight

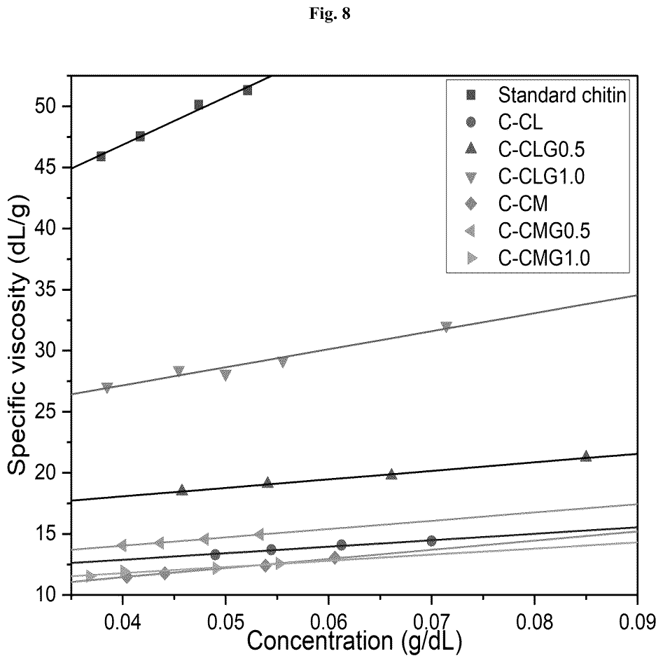

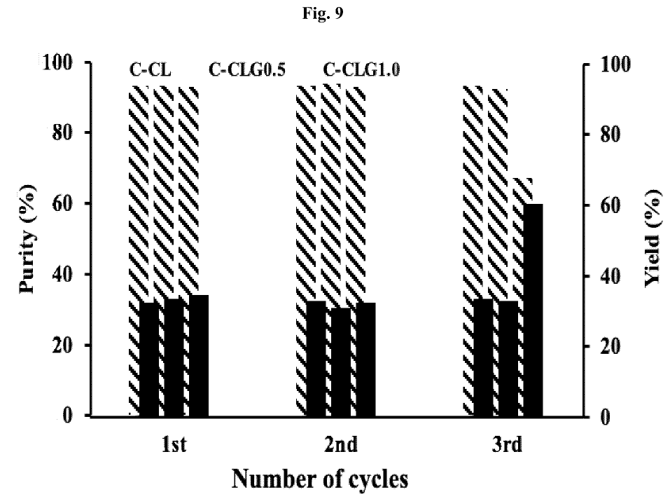

Molecular weight was calculated through intrinsic viscosity by recording the time of solution flowing out of the capillary. A higher molecular weight leads to increased intrinsic viscosity and increased time for flowing out. Chitin separated by glycerol-organic acid-based DES exhibited a low intrinsic viscosity and Mw when compared with standard chitin (FIG. 8 and Table 3). The organic acid and glycerol ratio has an effect on chitin molecular weight by influencing its partial hydrolysis degree under high concentration of acidic conditions. For lactic acid as HBD, the molecular weight of chitin follows the same order as lactic acid to glycerol ratio. The results revealed that decreasing the molar ratio of lactic acid to glycerol lowered the acid hydrolysis degree. When malic acid acted as HBD, the depolymerization degree increased even though the molar ratio of choline chloride to malic acid was reduced to 1:1 compared with the 1:2 molar ratio of choline chloride to lactic acid. This is in accordance with previous study reporting the malic acid has a stronger ability of depolymerization than lactic acid. However, when considering the glycerol addition, C-CMG1.0 showed a relatively small molecular weight which could be explained by the relative low purity of C-CMG1.0. the high protein residue in C-CMG0.5 could be easily dissolved into DMAc but showed a very low molecular weight, thus reducing the chitin molecular weight.

TABLE 3
intrinsic viscosity, weight average molecular weight and R-square for chitin samples.

| Sample | | | | | | | Standard |
|---|---|---|---|---|---|---|---|
| code | C-CL | C-CLG0.5 | C-CLG1.0 | C-CM | C-CMG0.5 | C-CMG1.0 | chitin |
| Intrinsic | 10.75 | 15.28 | 21.25 | 8.43 | 11.31 | 9.79 | 31.27 |
| viscosity | | | | | | | |
| (dL/g) | | | | | | | |
| M_{w} (kDa) | 264 | 382 | 541 | 204 | 278 | 239 | 881* |
| R² | 1.00 | 0.9952 | 0.9528 | 0.9922 | 0.9978 | 0.9445 | 0.9865 |

*the Mw of standard chitin has been out of range (80 kDa-710 kDa)

### TDES Recycling Performance

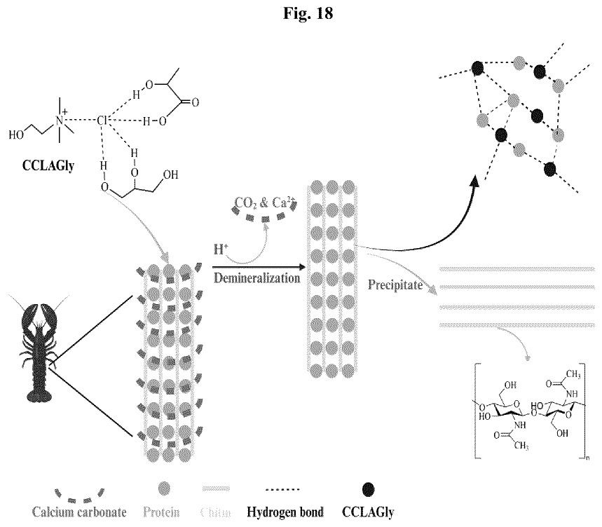

The recyclability of DES is of vital importance to fully evaluate DES performance. In the present study, both types of TDESs were employed to assess their chitin extraction effectiveness after recycling. However, when it came to malic acid-based TDESs, CCMAGly0.5 and CCMAGly1.0 exhibited increased viscosity and precipitation after a single cycle (data not shown). This phenomenon may be attributed to the dissolution of proteins from snow crab shells within the solution and the low solubility of calcium malate. Unlike malic acid-based TDESs, C-CLG1.0 also has a low purity after three cycles. The possible reason could be that the acidity of this solvent is too low to react with calcium carbonate after two cycles, which was further proved by the high mineral residue amount in the extracted chitin (28.85%). However, the other two lactic acid-based DESs, CCLA and CCLAGly0.5, demonstrated excellent recycling performance. As depicted in FIG. 18, even after three recycles, both purities remained high at 92.71% and 92.12%, respectively (dashed bars). The slight decrease in purity could be attributed to impurities in the regenerated solutions affecting hydrogen bonding, thereby reducing the processing efficiency of these two TDESs. Furthermore, no appreciable changes (from 31.86% to 34.16%) in the yields (solid bars) of the above two lactic acid based DESs, indicating their promising potential in chitin extraction.

As disclosed herein, glycerol was added into DES systems to lower the acidic conditions for chitin recovery with higher molecular weight. Chemical composition analysis, FTIR and XRD results proved the effective removal of protein and minerals from crab shell waste with varied organic acid content. Organic acid and glycerol molar ratios mainly affected the molecular weight, morphology and rheological behavior of extracted chitins. Chitin extracted by the DES system consisting of chloride, lactic acid and glycerol (CCLAGly1.0) had highest molecular weight about 541 kDa than other treatments which is explained by the less acid environment when glycerol is included. Malic acid was proved to have strong depolymerization and more acid hydrolysis was observed on CCMA extracted chitin. Lactic acid-based TDES showed excellent deproteinization and demineralization ability even after three times reuse. Therefore, TDES composed of lactic acid showed prominent potential in extracting chitin from crab shell waste and meeting the demand for sustainability and economic efficiency.

### Example 2

### Introduction

The disposal of lobster shells (LS), a byproduct of seafood processing, raises a huge problem for food industries. It was reported that approximately 40-50% of the total mass of lobster is waste. Generally, LS are either discarded into the sea or dumped in landfills along the shoreline, surpassing the rate of natural recycling processes. This excessive waste disposal not only emits foul odors but also releases biogenic amides during decay, posing a threat to the ecological balance of coastal areas. The waste of LS contains several chemicals, such as protein, calcium carbonate, chitin, and astaxanthin, which are often underutilized. Among these components, chitin stands out as a natural polymer abundantly present in LS, constituting 20-30% of their composition. Chitin holds great potential for numerous industrial applications, particularly in the pharmaceutical and agricultural sectors. Hence, finding a suitable method to extract chitin from LS is of great importance.

The traditional chemical method of chitin extraction typically needs strong acids and bases to eliminate protein and calcium carbonate in LS. While this method could extract chitin with desirable purity, it generates a significant amount of acidic and alkaline effluent, posing substantial ecological risks and leading to environmental pollution. Numerous endeavors have been dedicated to discovering innovative methods for environmentally sustainable chitin extraction. Among these, deep eutectic solvents (DESs) have emerged as a promising alternative. DESs were first introduced in 2003 (A. P. Abbott, et al., Novel solvent properties of choline chloride/urea mixtures, Chemical communications (1) (2003) 70-71). These solvents generally consist of two or three components capable of forming a eutectic mixture with a lower melting point compared to any single component due to the interaction among these components. The above components can be categorized into two groups:hydrogen bond acceptors (HBAs) and hydrogen bond donors (HBDs). Among the HBAs investigated, choline chloride has gained considerable attention due to its low toxicity, low cost, biodegradability, and biocompatibility (P. Suthar, et al., Deep eutectic solvents (DES): an update on the applications in food sectors, Journal of Agriculture and Food Research 14 (2023) 100678; M. Sainakham, et al., Potential of green extraction using edible deep eutectic solvents on the bioactivities from Curcuma aromatica rhizome extracts for food application, Journal of Agriculture and Food Research 14 (2023) 100868). Meanwhile, HBDs usually comprise carbohydrates, polyols, carboxylic acids, and other similar compounds.

Wang, et al., investigated the interaction in DESs composed of choline chloride and polyols [H. Wang, et al., Insights into the hydrogen bond interactions in deep eutectic solvents composed of choline chloride and polyols, ACS sustainable chemistry & engineering 7 (8) (2019) 7760-7767]. Their results revealed that the predominant interaction was hydrogen bonding between the chlorine atom of choline chloride and the hydrogen atom of the OUH group in the polyols. Additionally, they noted that the strength of this interaction decreased with the decrease in the number of hydroxyl groups in polyols. Chitin in LS is commonly intertwined with proteins, primarily through hydrogen bonds, and reinforced by calcium carbonate. Acidic DESs offer both hydrogen bonds and hydrogen ions, facilitating the simultaneous removal of proteins and calcium carbonate from the chitin structure. Previously, lactic acid- and malic acid-based DESs were developed for extracting chitin from snow crab shells. Both types of acidic DESs exhibited high extraction efficiencies, yielding chitin with purities exceeding 95%.

In another study, Rodrigues, et al., demonstrated the feasibility of utilizing choline chloride/lactic acid-based DES to extract pure chitin, with purity reaching up to 98% from brown crab shells (L. A. Rodrigues, et al., Low-phytotoxic deep eutectic systems as alternative extraction media for the recovery of chitin from brown crab shells, ACS omega 6 (43) (2021) 28729-28741).

Inventors investigated DESs composed of choline chloride as the HBA, and lactic acid, along with polyols (ethylene glycol, glycerol, xylitol, and sorbitol) as HBDs. The analysis focused on examining the hydrogen bond interactions among these components using Fourier-transform infrared spectroscopy (FTIR), rheometer measurements, and molecular modeling techniques. Additionally, the efficacy of these DESs was explored in extracting chitin from LS, followed by a comparative analysis of the purity, molecular weight, and physicochemical properties of extracted chitin. The structure and properties of extracted chitin were further characterized using FTIR, X-ray diffraction (XRD), thermogravimetric analysis (TGA), and scanning electron microscopy (SEM).

### Materials and Methods

### Materials

Lobster shells (LS) were kindly provided by East Coast Seafood Company (New Bedford, MA, USA). The LS was washed with hot water and then removed all the meat and other impurities. LS was subsequently dried in the oven at 70° C. overnight and then ground using a kitchen grinder to obtain powder size with 60 mesh. Commercial chitin and all other chemicals were purchased from Thermo Fisher Scientific. N, N-Dimethylacetamide (DMAc) and lithium chloride (LiCl) were obtained from Acros-Organics.

### Synthesis of Deep Eutectic Solvent (DES)

The DESs were prepared by mixing hydrogen bond acceptors (HBA) with hydrogen bond donors (HBD) in a fixed molar ratio at 50° C. until transparent and homogeneous liquids were formed (See Table 4 for formulation details).

TABLE 4
List of the prepared DES used in this work.

| | | | Molar ratio | |
|---|---|---|---|---|
| Abbreviation | HBA | HBD | (HBA:HBD) | Appearance |
| CCLaEg | Choline | Lactic acid + | 1:1:1 | Transparent |
| | chloride | Ethylene Glycol | | liquid |
| CCLaGly | Choline | Lactic acid + | 1:1:1 | Transparent |
| | chloride | Glycerol | | liquid |
| CCLaPen | Choline | Lactic acid + | 1:1:1 | White |
| | chloride | Pentaerythritol | | solid |
| CCLaXyl | Choline | Lactic acid + | 1:1:1 | Transparent |
| | chloride | Xylitol | | liquid |
| CCLaSor | Choline | Lactic acid + | 1:1:1 | Transparent |
| | chloride | Sorbitol | | liquid |

### Characterization of DES

The chemical structures and internal interactions of the prepared DESs were determined by FTIR spectroscopy and the rheometer, and the molecular modeling. The FTIR spectra of choline chloride, lactic acid, ethylene glycol, glycerol, xylitol, and sorbitol were obtained using the Fisher Scientific Nicolet iS5 Spectrometer equipped with an iD7 Diamond ATR accessory (Thermo Fischer Scientific Inc., Waltham, MA, USA) within the wavenumber range of 500-400 cm⁻¹ and analyzed by OMNIC software version 8.0. The viscosity of all prepared DESs was evaluated by a rheometer (MCR 302, Anton Paar, Graz, Austria) equipped with double-gap concentric cylinder measurement system. The samples with around 0.5 mL loaded between the concentric cylinders with a gap of 0.1 mm were performed with shear mode (0.01-50 s-1) at 50° C. The calculation of hydrogen bonds in DESs was conducted through the Materials Studio software package. The simulations were all conducted using the COMPASS force field. Long-range electrostatic interactions and van der Waal interactions were calculated using the Ewald and atom-based methods, respectively, with a cutoff radius of 1.25 nm. An NVT ensemble was chosen for the dynamic simulation of the solution, with the Nosé method used for temperature control. The time step was set to 1.0 fs, and the total simulation duration was 1 ns, with the first 500 ps used to equilibrate the system and the remaining 500 ps used to calculate hydrogen bonds.

### Chitin Extraction

The LS and DES solutions were mixed at a mass ratio of 1:20 and heated at 50° C. for 2 h under magnetic stirring. The mixture was cooled to room temperature with the addition of distilled water. Then, it was filtered under vacuum, and the solids were washed with distilled water until reaching a neutral pH. Finally, the extracted chitin was dried overnight at 70° C. and stored in a desiccator for further use. The extracted chitins were designated as CCLaEg-Chitin, CCLaGly-Chitin, CCLaXyl-Chitin, and CCLaSor-Chitin.

Compared to the chitins extracted by DESs, the chemical isolation of chitin was conducted with some modifications according to the method described by Zhu at al., (P. Zhu, et al., One-pot production of chitin with high purity from lobster shells using choline chloride-malonic acid deep eutectic solvent, Carbohydrate polymers 177 (2017) 217-223). Firstly, 1.5 g of LS was treated with 3% hydrochloric acid (1:25 w/v) at 25° C. for a two-hour demineralization process. Secondly, the samples were filtered, washed with distilled water, and dried overnight at 70° C. Thirdly, the dried samples were treated with 10% sodium hydroxide (NaOH) (1:15 w/v) under stirring for 3 h at 90° C. to remove protein. At last, the final samples were filtered, washed, dried overnight, and stored in the desiccator. The chemical-isolation chitin was designated as Chem-Chitin.

### Chemical Composition Analysis

### Ash and Protein Content

The ash content was measured gravimetrically using thermalgravimetric analysis (TGA, TA instruments Q500-0188, USA). Around 12 mg of the sample was heated at a rate of 10° C./min, held at 105° C. for 30 min, and then continued heating until reaching 800° C. for 30 min under an air atmosphere. The protein content was determined using the Lowry method with Bovine serum albumin as a standard. In brief, 50 mg of the sample was pre-treated by 10 mL of 5% NaOH solution, then heated and stirred at 95° C. for 2.5 h. Next, the mixture was centrifuged to collect supernatant for protein measurement. The protein content was estimated by measuring the absorbance at 562 nm. Yield and purity were calculated based on Eqs. (1) and (2):

$$Yield = \frac{{W}_{1}}{{W}_{0}} \times 100 \% \qquad ( 1 )$$

$$Purity = \frac{{W}_{1} - {W}_{pro} - {W}_{mineral}}{{W}_{1}} \times 100 \% \qquad ( 2 )$$

Where W₀ and W₁ were the initial weight of LS and the weight of extracted chitin, respectively. Wpro and Wmineral were the weights of residual protein and mineral content in extracted chitin.

### Molecular Weight Measurement

The viscosity-based measurement of molecular weight (M_{w}) was conducted using a Ubbelohde capillary viscometer with a diameter of Φ=0.4-0.5 mm at 2θ° C. Before testing, different amounts of chitin samples were dissolved in 5% (w/w) DMAc/LiCl solvents under magnetic stirring overnight. The intrinsic viscosity value was estimated by the Huggins and Kraemer Eqs. (3) and (4):

$$Huggins : \frac{{η}_{s p}}{c} = [ η ] + {{k}^{′} [ η ]}^{2} c \qquad ( 3 )$$

$$Kraemer : ( ln {η}_{r} ) / c = [ η ] + {{k}^{″} [ η ]}^{2} c \qquad ( 4 )$$

Where k′ and k″ represent the constants depending on the molecular properties, experimental conditions and solvent; ηₛₚ/c denotes the reduced specific viscosity; (Inηᵣ)/c was the inherent viscosity; c was the chitin concentration.

The intrinsic viscosity obtained was applied to the Mark-Houwink-Sakurada Eq (5) to estimate the Mw of chitin, which falls within the range of 80 to 710 kDa.

$$[ η ] = 7.6 \times {10}^{- 5} {M}_{W}^{0.95} \qquad ( 5 )$$

### Characterization of Isolated Chitin

### FTIR

The FTIR spectroscopy of LS, commercial chitin, Chen-Chitin, and DES-extracted chitin were conducted as in paragraphs [0128] and [129].

### X-Ray Transform Infrared Spectroscopy (XRD)

XRD analysis was performed on a Bruker D2 Phaser Diffractometer (Bruker, Germany). The 2θ was in a range of 5°-55° with a step size of 0.01° and 2 s/step as the counting time. The crystallinity index (CrI; %) was calculated according to the following Eq. (6):

$$C r {I}_{110} = [ \frac{{I}_{110} - {I}_{am}}{{I}_{110}} ] \times 100 \qquad ( 6 )$$

Where I₁₁₀ is the maximum intensity at 2θ≅19° and Iₐₘ is the intensity of amorphous diffraction at 2θ≅12.6°.

### TGA

TGA curves were analyzed using the TA instruments Q500-0188 analyzer over a temperature from 30 to 700° C., with a heating rate of 10° C./min under nitrogen. Data was recorded from 100 to 700° C.

### Scanning Electron Microscope (SEM)

SEM (JSM-6335F, JOEL Ltd., Japan) was employed to observe the surface morphology of LS, commercial chitin, Chem-Chitin, and DES-extracted Chitin under an accelerating voltage of 2 kV after gold-coated by a sputter coater.

### Statistical Analysis

All statistical analyses were performed using IBM SPSS Statistics for Windows version 23.0 (SPSS Inc., USA). One-way ANOVA followed by the Duncan test was performed to determine the significance of difference at P<0.05 among tested groups. All experiments were conducted in triplicate unless otherwise stated.

### Results and Discussion

### DES Formation

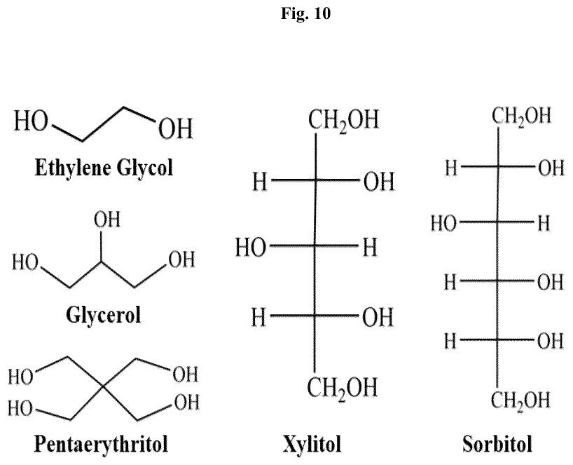

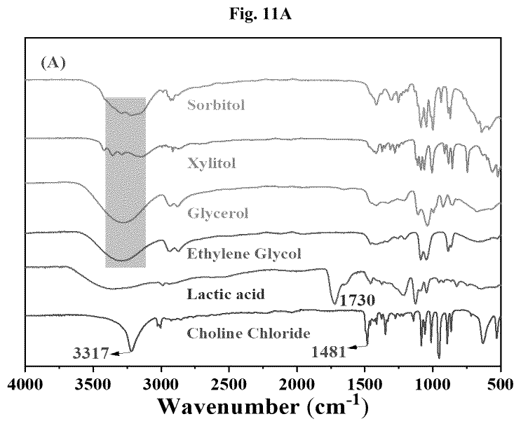

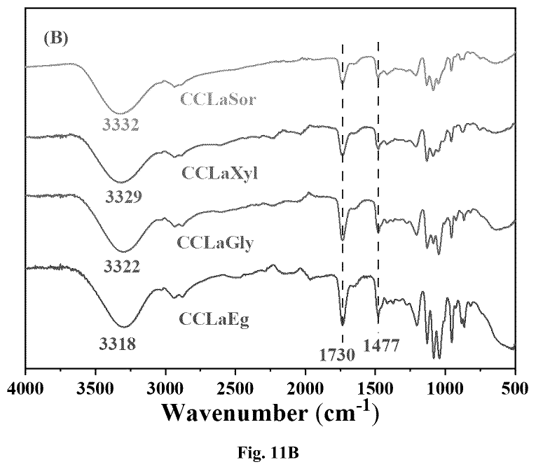

The most commonly used method for synthesizing DES involves heating and stirring until a homogenous liquid mixture is formed. All molecular structures of five selected polyols were shown in FIG. 10. CCLaEg, CCLaGly, CCLaXyl, and CCLaSor DESs were successfully prepared, each displaying a clear and homogeneous state at room temperature. However, precipitation was observed in the CCLaPen DES, which was unsuitable for further use and discarded. In this context, choline chloride serves as the HBA and can form hydrogen bonds with the carboxyl and hydroxyl groups on lactic acid and polyols. The viscosities of prepared DESs increased with the growing number of hydroxyl groups, likely due to the formation of more hydrogen bonds. These hydrogen bonds present in the DES system facilitate the separation of chitin by disrupting the original hydrogen bonds in LS. However, it was noteworthy that high viscosity would hinder chitin extraction efficiency. Further in-depth characterizations of prepared DESs were analyzed and discussed below.

### DES Characterization

### Chemistry and Composition of the DESs

The FTIR and rheometer were employed to analyze the potential interaction among the internal functional groups. The spectra of choline chloride, lactic acid, polyols, and prepared DESs were shown in FIG. 11. In FIG. 11A, the peaks observed around 3317 cm⁻¹ and 1481 cm⁻¹ corresponded to the O—H stretching vibration and CH2 bond bending vibration of choline chloride, respectively. Besides, the peak at 1730 cm⁻¹ was attributed to C—O stretching vibration, characteristic of lactic acid. For the polyols, a broad peak around 3300-3200 cm⁻¹ was observed due to the O—H stretching vibration. FIG. 11B showed the O—H stretching vibrations in CCLaEg, CCLaGly, CCLaXyl, and CCLaSor, measured at 3318, 3322, 3329, and 3332 cm⁻¹, respectively. The O—H stretching frequency of the prepared DESs exhibited a slight blue shift when compared with choline chloride and polyols. This shift can be ascribed to the breaking of intermolecular hydrogen bonds within choline chloride itself upon the addition of polyols, leading to stronger hydrogen bond interactions between choline chloride and polyols. Notably, the shift of choline chloride from 1481 to 1477 cm⁻¹ was consistent with previous research (R. Ninayan, et al., Water-induced changes in choline chloride-carboxylic acid deep eutectic solvents properties, Colloids and Surfaces A: Physicochemical and Engineering Aspects 679 (2023) 132543).

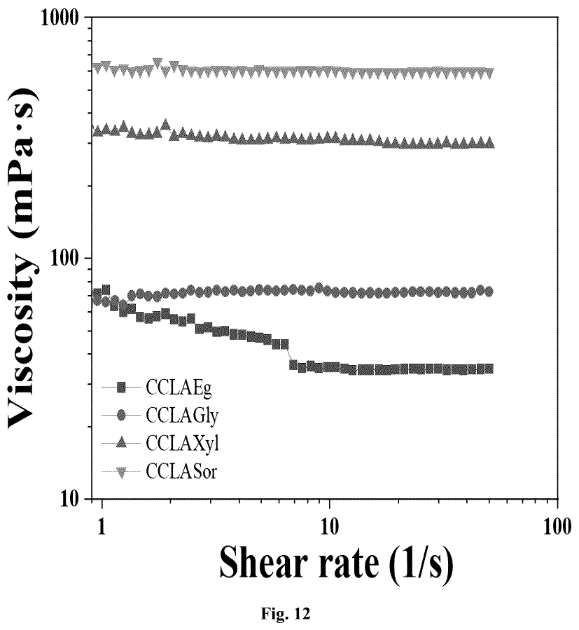

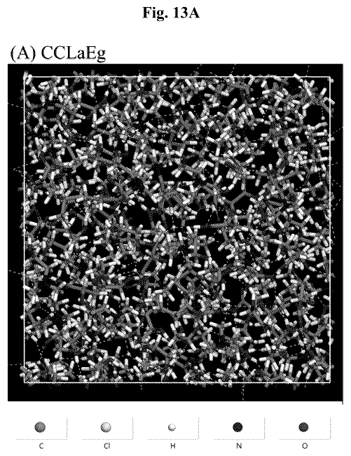

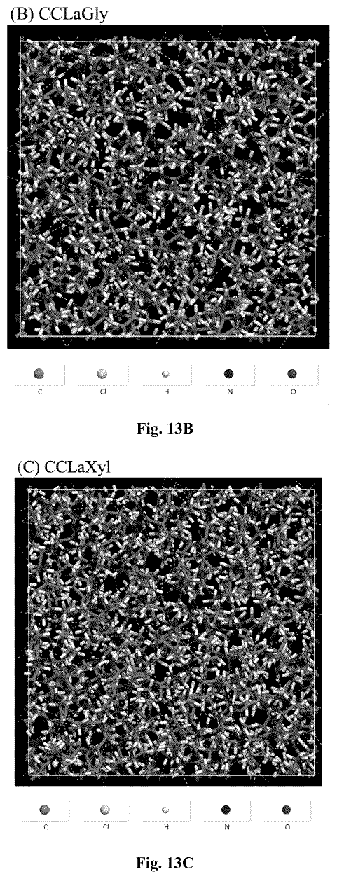

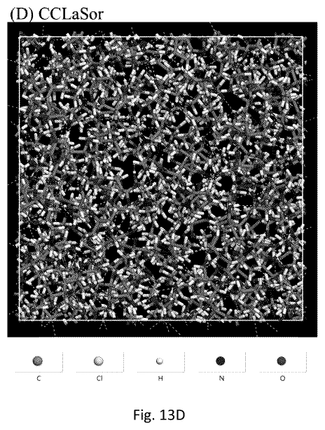

Rheological properties of prepared DESs were assessed using a rheometer under shear mode. As shown in FIG. 12, CCLaGly, CCLaXyl, and CCLaSor exhibited Newtonian fluid behavior, characterized by constant viscosity independent of the applied shear rate value and duration, indicating the absence of molecular structuring or entanglements. However, the shear thinning was observed in CCLaEg from 0.1 to 10 S-1, followed by a transition to Newtonian fluid behavior between 10 and 100 S-1. In detail, during the initial stages of shear thinning behavior, molecules in CCLaEg may rearrange under shear force, resulting in a sudden decrease; once this rearrangement reaches a certain extent, CCLaEg may resemble a Newtonian fluid, where viscosity remains independent of shear rate. This phenomenon could arise from molecules in CCLaEg beginning to align along the flow direction under shear force, with sufficiently strong interactions to maintain stable viscosity within a certain range. The viscosity values of CCLaEg, CCLaGly, CCLaXyl, and CCLaSor were determined to be 34.76, 72.45, 299.22, and 596.09 mPa·s, respectively. With an increase in the number of hydroxyl groups in polyols, the viscosity of DESs demonstrated a corresponding increase. This phenomenon can be explained by the formation of a larger network of hydrogen bonds between choline chloride and polyols with more hydroxyl groups, consequently limiting molecular mobility and resulting in increased viscosity.

### Molecular Modeling Results

The ball-and-stick models of prepared DESs were simulated using Materials Studio software. As depicted in FIG. 13, evident intermolecular hydrogen bonds were observed between choline chloride and lactic acid, as well as the polyols. Specifically, distinct hydrogen bond interactions were noted between the chlorine atom of choline chloride and the hydrogen atom from the hydroxyl and carboxylic groups of lactic acid and polyols. Additionally, the theoretical number of hydrogen bonds present in CCLaEg, CCLaGly, CCLaXyl, and CCLaSor was calculated, which was around 278, 351, 482, and 553, respectively. This result indirectly suggests a denser hydrogen bond network forming with an increase in the number of hydroxyl groups in the polyols, corroborating the findings from the rheological experiment. The formation of a denser hydrogen bond network could significantly affect the physical properties of DESs, such as viscosity and solubility. With more hydrogen bonds formed, the interactions between the components became stronger, potentially leading to increased viscosity and altered solvation behavior. This phenomenon aligns with the observed trend in the rheological data, where viscosity increased with the number of hydroxyl groups in the polyols. Thus, computational analysis of hydrogen bond networks provided valuable insight into the structure-property relationships of prepared DESs, helping better understand the mechanism of chitin extraction.

### Chemical Composition Analysis of the Extracted Chitin

To study the purity of chitin extracted by CCLaEg, CCLaGly, CCLaXyl, and CCLaSor DESs, the main chemical compositions alongside those of Chem-Chitin, commercial chitin, and LS were analyzed and compared based on dried weight. According to Table 5, the moisture contents of all the extracted chitins ranged from 2.84 to 4.23%, which was lower than that of lobster shells (5.44%). Besides, CCLaSor-Chitin exhibited the highest yield at 35.55±1.68%, and Chem-Chitin showed the lowest yield at 16%. However, the yield is not the main factor of the extraction. Instead, the ash and protein content are crucial indicators of chitin purity. Compared with LS, the ash content in samples of CCLAEg-Chitin, CCLAGly-Chitin, CCLAXyl-Chitin, and CCLASor-Chitin was reduced to 16.76±2.25%, 1.23±0.28%, 9.5±1.94%, and 20.55±0.01%, respectively (P<0.05). It was noted that only CCLaGly-Chitin displayed superior efficacy in removing calcium carbonate and other minerals from LS, suggesting that CCLaGly DES is particularly effective for chitin extraction. In addition, among the CCLaEg-Chitin, CCLaGly-Chitin, CCLaXyl-Chitin, and CCLaSor-Chitin, no significant differences (P>0.05) were observed in protein content (4.13±0.26%, 4.01±0.04%, 3.95±0.15%, and 3.64±0.23%, respectively), consistent with previous findings. Furthermore, the purity of Chem-Chitin matched that of commercial chitin and surpassed that of DES-extracted chitin. This discrepancy in purity could be attributed to the properties of DESs, as their viscosity may impede penetration into LS, resulting in incomplete removal of protein and calcium carbonate and subsequently lowering the purity of the extracted chitin.

The molecular weights of all prepared samples were determined based on intrinsic viscosity, calculated by recording the time for the chitin solution to flow out of the capillary. Higher molecular weights result in increased intrinsic viscosity and longer flow-out time. Compared with commercial chitin, only CCLaGly-Chitin and Chem-Chitin exhibited molecular weights close to it. A previous study demonstrated that reducing the molar ratio of lactic acid to glycerol to 1:1 decreased the degree of acid hydrolysis, leading to a higher molecular weight (Y. Wang, H. et al., Glycerol/organic acid-based ternary deep eutectic solvents as a green approach to recover chitin with different molecular weight from seafood waste, International Journal of Biological Macromolecules 257 (2024) 128714). However, for the high molecular weight of Chem-Chitin, the possible explanation could be the low reaction temperature and short reaction time in acidic conditions. Regarding CCLaEg-Chitin, CCLaXyl-Chitin, and CCLaSor-Chitin, their molecular weights decreased with their purity. Impurities in the extracted chitin significantly affect its concentration in DMAc/LiCl solvent, thereby reducing intrinsic viscosity and ultimately lowering the molecular weight.

TABLE 5
Characteristics of DES-extracted, Chem-extracted, and commercial chitin,
as well as the chemical composition of original lobster shells.

| | CCLaEg- | CCLaGly- | CCLaXyl- | CCLaSor- | Chem- | Commercial | |
|---|---|---|---|---|---|---|---|
| Sample | Chitin | Chitin | Chitin | Chitin | Chitin | Chitin | LS |
| Yield | 28.89 ± | 26.22 ± | 26.67 ± | 35.55 ± | 16 ± | — | — |
| (%) | 1.54^{b} | 2.14^{b} | 0.00^{b} | 1.68^{c} | 0.00^{a} | | |
| Moisture | 4.09 ± | 3.43 ± | 4.23 ± | 2.84 ± | 3.56 ± | 4.68 ± | 5.44 ± |
| (%) | 1.07^{ab} | 0.18^{ab} | 1.00^{ab} | 0.29^{a} | 0.38^{ab} | 0.08^{bc} | 0.11^{c} |
| Protein | 4.13 ± | 4.01 ± | 3.95 ± | 3.64 ± | 0.83 ± | 0.86 ± | 10.35 ± |
| (%) | 0.26^{b} | 0.04^{b} | 0.15^{b} | 0.23^{b} | 0.02^{a} | 0.05^{a} | 0.22^{c} |
| Ash (%) | 16.76 ± | 1.23 ± | 9.5 ± | 20.55 ± | 0.94 ± | 0.90 ± | 49.85 ± |
| | 2.25^{c} | 0.28^{a} | 1.94^{b} | 0.01^{d} | 0.21^{a} | 0.49^{a} | 1.17^{e} |
| Purity | 79.22 ± | 94.76 ± | 86.50 ± | 75.45 ± | 98.23 ± | 98.24 ± | — |
| (%) | 2.02^{b} | 0.33^{d} | 1.79^{c} | 0.56^{a} | 0.20^{e} | 0.53^{e} | |
| Intrinsic | 20.53 | 25.50 | 21.76 | 11.17 | 27.37 | 31.27 | — |
| viscosity | | | | | | | |
| (dL/g) | | | | | | | |
| M_{w} | 522 | 655 | 555 | 275 | 706 | 881 | — |
| (kDa) | | | | | | | |

Different lower-case letters indicate significant differences among groups (P < 0.05).

### FTIR

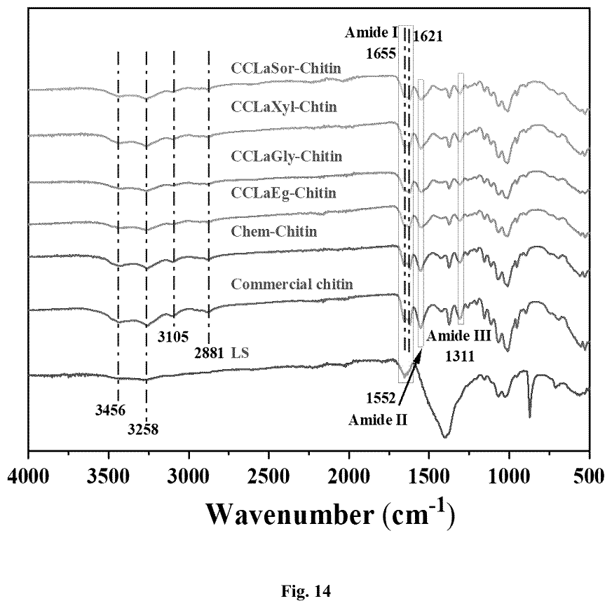

The FTIR spectra of the DES-extracted chitin, Chem-Chitin, commercial chitin, and LS are shown in FIG. 14. In these spectra, the broad bands at 3456 and 3258 cm⁻¹ corresponded to O—H and N—H stretching, respectively. Additionally, the bands ranging from 3105 to 2881 cm⁻¹ represent CH, CH3 symmetric stretching and CH2 asymmetric stretching. Furthermore, the amide I exhibited a split at 1655 cm−1 and 1621 cm−1, indicating two types of hydrogen bonding: one between the NH of the adjacent chain (CO . . . HN) and N-acetyl carbonyl group, and the other between the carbonyl and the primary hydroxyl group of the same chain (CO . . . HOCH2). It was worth noting that this splitting phenomenon was absent in the LS, likely due to protein amide peaks overlapping with the chitin amide I band, suggesting that DESs can effectively remove protein from LS. Moreover, the presence of amide II at 1552 cm⁻¹ and amide III at 1311 cm−1 were also noted on the spectra. Overall, the structural analysis shown in FIG. 14 revealed the chitin obtained from both DES and the chemical method closely resembles commercial chitin, indicating that DES, like traditional chemical methods, facilitated successful deproteinization reactions.

### XRD

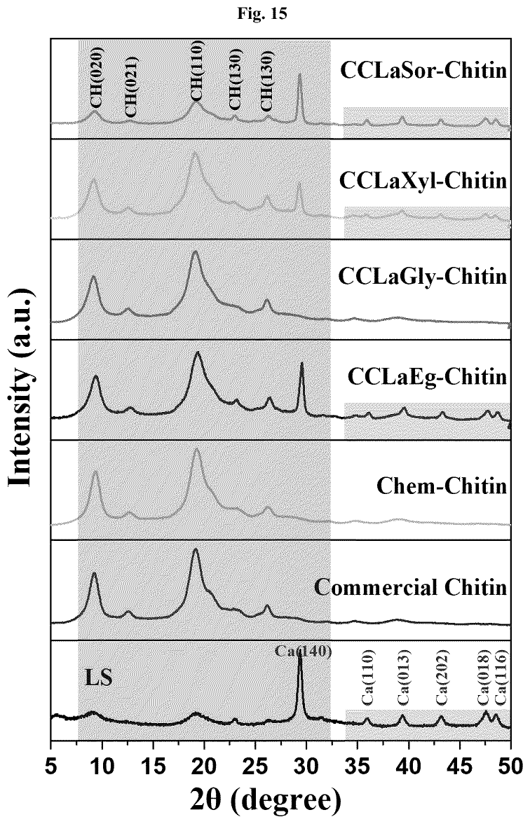

The XRD patterns of LS, commercial chitin, Chem-Chitin, and DES-extracted chitin were displayed in FIG. 15. In the spectra of commercial chitin, Chem-Chitin, and DES-extracted chitin, two broad crystalline reflections at 9.2° and 19.2°, along with pale diffraction peaks at 12.65°, 23.41°, and 26.29°, indicated the presence of a stable α-chitin structure. However, in the case of LS, six diffraction peaks were observed, notably the peak at 2θ≅29.8°, characteristic of calcium carbonate. Raabe, et al., provided a detailed description of the hierarchy of the main structural and microstructural elements of lobster shells (D. Raabe, et al., The crustacean exoskeleton as an example of a structurally and mechanically graded biological nanocomposite material, Acta Materialia 53 (15) (2005) 4281-4292). They explained that calcium carbonate is embedded within the chitin-protein fiber that creates a planar woven and periodically branched network. Comparing all the DES-extracted chitins, only CCLaGly-Chitin exhibited good agreement with Chem-Chitin and commercial chitin, with a chitin purity of around 95% and negligible calcium carbonate content (1.23±0.28%). It was inferred that glycerol in the DES system could provide enough hydrogen bonds to break the chitin-protein fiber, and then hydrogen ions from the lactic acid could easily remove the exposed calcium carbonate. Conversely, CCLaEg-Chitin, CCLaXyl-Chitin, and CCLaSor-Chitin showed all six peaks of calcium carbonate, consistent with their higher calcium carbonate contents (16.76±2.25%, 9.5±1.94%, and 20.55±00.01%, respectively). The lower effectiveness could be due to the formation of fewer hydrogen bonds in CCLaEg DES and the higher viscosity in CCLaXyl and CCLaSor DESs, which are difficult to thoroughly destroy the chitin-protein fiber, hindering the exposure of calcium carbonate to hydrogen ions.

The crystallinity indexes of LS, commercial chitin, Chem-Chitin, CCLaEg-Chitin, CCLaGly-Chitin, CCLaXyl-Chitin, and CCLaSor-Chitin were calculated as follows: 31.15%, 80.58%, 78.78%, 72.11%, 77.73%, 73.29%, and 67.5%. The increase in chitin crystallinity positively correlated with chitin purity, suggesting the successful removal of both calcium carbonate and proteins from the matrix. Furthermore, CCLaGly DES yielded chitin with a crystallinity index comparable to that of commercial chitin and Chem-Chitin.

### TGA

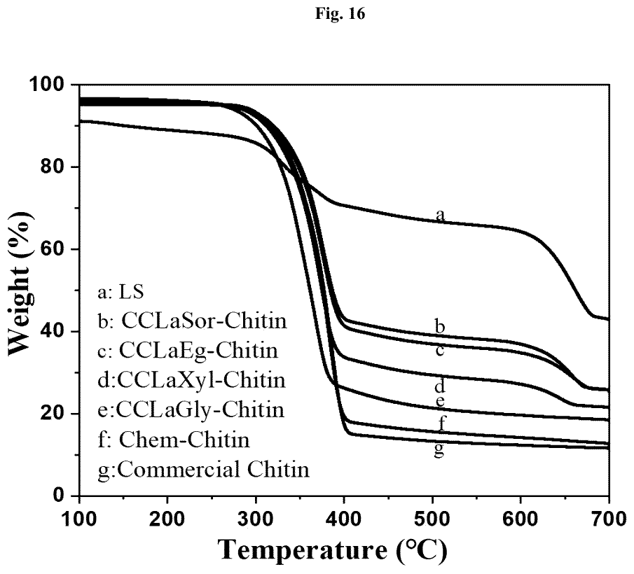

TGA analysis was conducted to examine the thermal degradation of samples. FIG. 16 showed TGA curves for the commercial chitin and DES-extracted chitin. Before 100° C. (data not shown), slight decomposition occurred due to the evaporation of chemisorbed water. The second stage, between 250° C. and 400° C., was attributed to chitin decomposition, where the chitin structure broke down into oligosaccharides. In LS, mass loss occurred from 100° C. to 250° C. due to protein and lipid breakdown. It was noted that the absence of mass loss in DES-extracted chitin between 100° C. and 250° C. indicated the successful removal of most protein and lipids by the DESs. Weight loss in LS occurred from 650° C. to 700° C., ascribed to calcium carbonate decomposition into carbon dioxide and calcium oxide. However, similar calcium carbonate decompositions were observed in CCLaEg-Chitin, CCLaXyl-Chitin, and CCLaSor-Chitin, suggesting these three DES formations were less suitable for chitin extraction from LS, consistent with XRD results. When it comes to CCLaGly-Chitin, its curve closely resembled that of commercial chitin and Chem-Chitin, indicating remarkably high chitin purity extracted using CCLaGly DES. Moreover, the extraction efficiency of this formulation was comparable to that of the chemical method.

### SEM

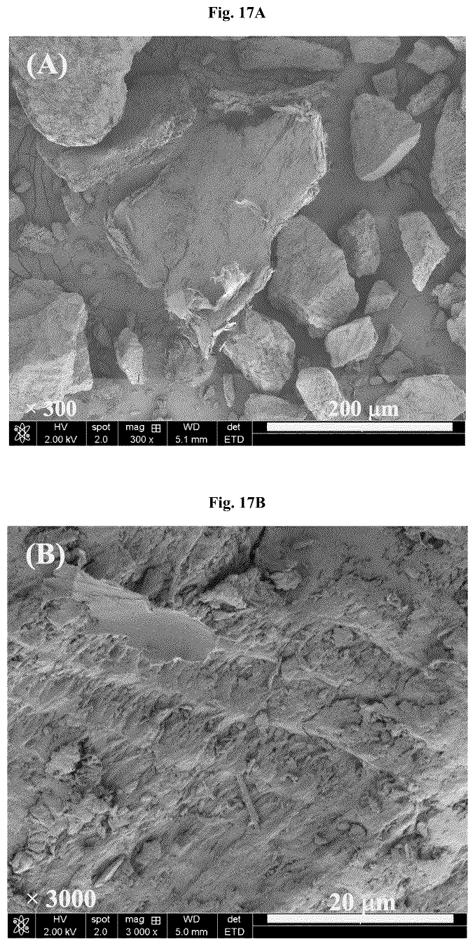

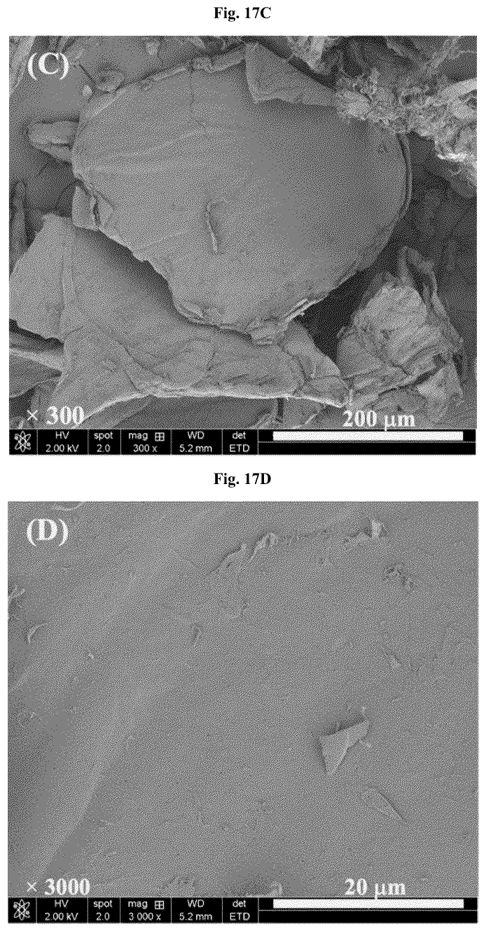

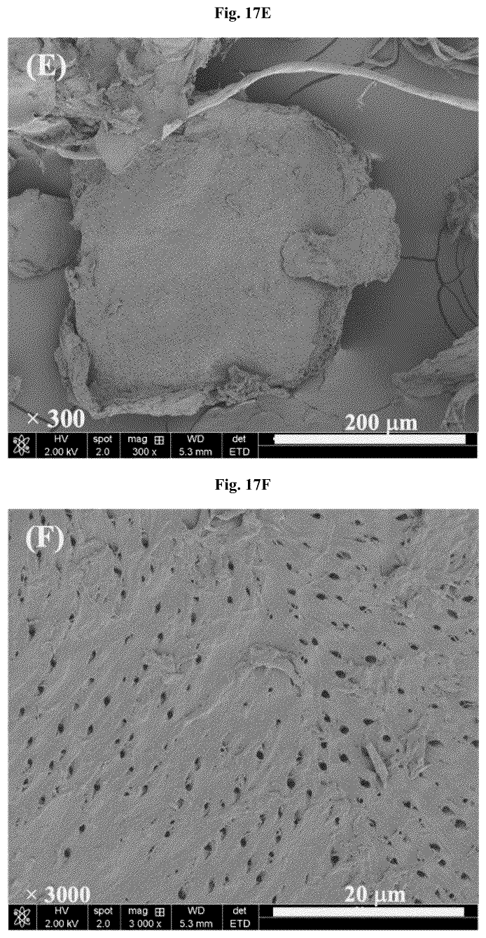

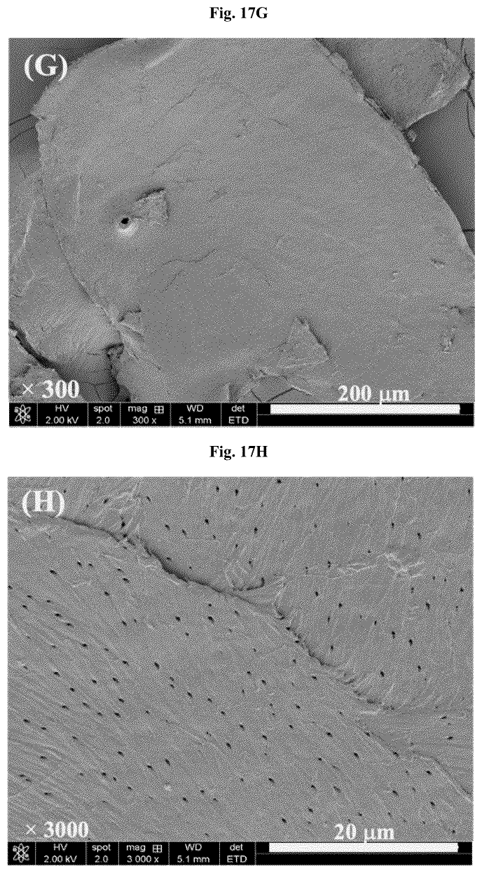

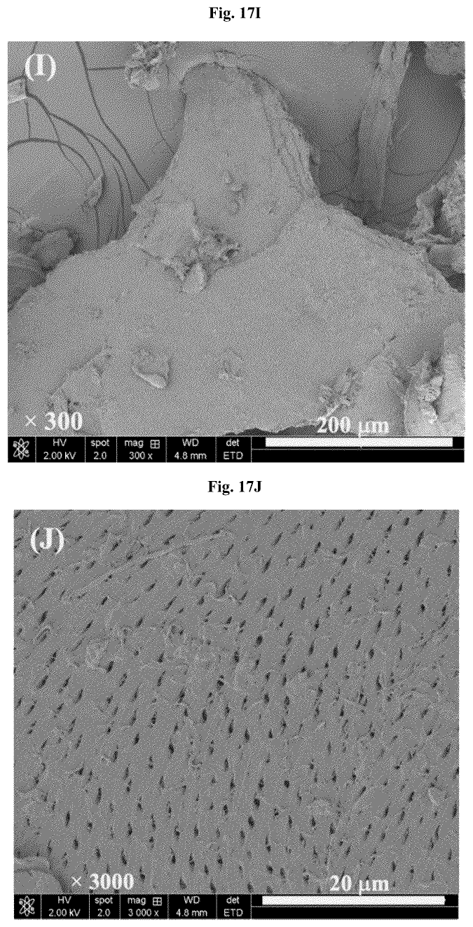

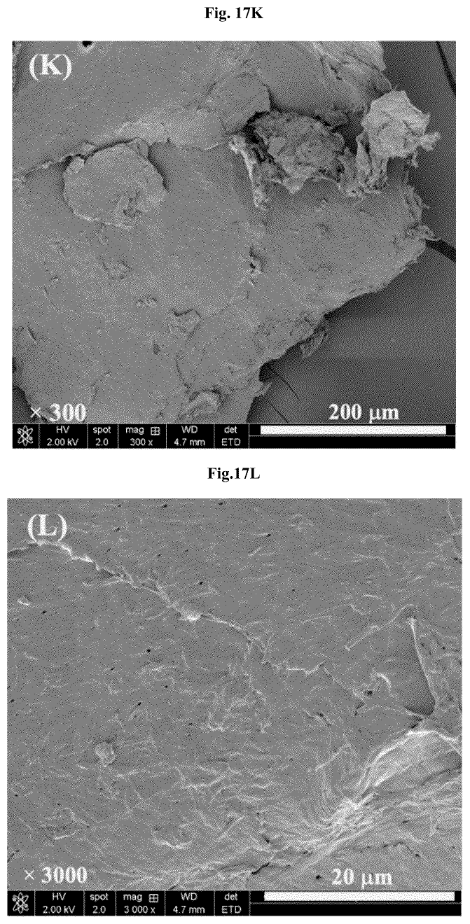

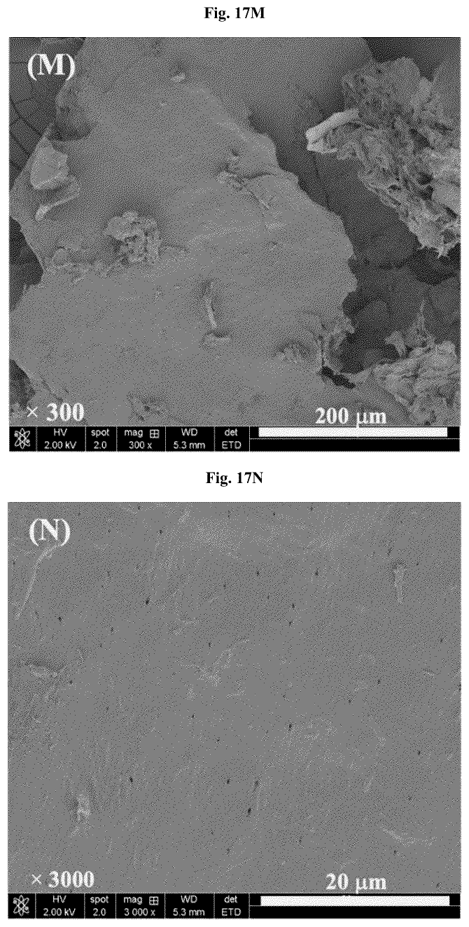

The SEM images in FIG. 17 illustrated LS, commercial chitin, and DES-extracted chitin, with different scale bars (200 μm and 20 μm). Chitin can be categorized into three groups based on their structure characteristics: (1) no porous and microfibrillar structure, (2) porous or microfibrillar structure, and (3) only microfibrillar structure. As shown in FIG. 17C and FIG. 17D, no porous and microfibrillar structure was observed in commercial chitin. However, the surface of LS (FIG. 17A and FIG. 17B) appeared rough and showed a multilayered accumulation morphology, likely attributed to impurities such as minerals. In contrast, the morphology of CCLaGly-Chitin (FIG. 17I and FIG. 17J) displayed high-density porous and fibrous structures, like those of the Chem-Chitin (FIG. 17E and FIG. 17F), indicating the superior capacity of CCLaGly DES to remove the ash and protein, again. Although the pores of CCLaEg-Chitin (FIG. 17G and FIG. 17H), CCLaXyl-Chitin (FIG. 17K and FIG. 17L), and CCLaSor-Chitin (FIG. 17M and FIG. 17N) were smaller and fewer compared to Chem-Chitin and CCLaGly-Chitin, they all exhibited smoother surfaces than LS, resembling commercial chitin. This suggests that proteins and minerals were partly removed during the extraction process.

### Mechanism of DES Chitin Extraction

Chitin in LS typically exists in the form of chitin-protein fibers and calcium carbonate is usually embedded in them. Therefore, it is crucial to remove proteins and calcium carbonate to obtain the desired chitin from LS. The schematic mechanism of the chitin extraction by DESs was illustrated in FIG. 18. On the one hand, the removal of calcium carbonate was achieved using lactic acid. The free hydrogen ions released by CCLa-based DESs engaged in the reaction. However, concerning CCLaXyl and CCLaSor DESs, the efficiency of calcium carbonate removal was significantly lower compared to that of CCLaGly. This difference could be ascribed to a rise in viscosity. Rheological studies demonstrated that viscosity increased with the number of hydroxyl groups. The elevated viscosity impedes the penetration of DESs into LS, leading to incomplete removal of calcium carbonate and consequently reducing the purity of the extracted chitin. On the other hand, due to partly removal of calcium carbonate by DESs, the linkages in the inner structural organization of LS were weakened because of the increased space between the chitin-protein fibers. The separation of protein from the protein-chitin fibers was facilitated due to the intermolecular and intramolecular hydrogen bonding present in the prepared DESs. In detail, proteins are rich in carboxylic and hydroxyl groups, serving as HBD that compete for chloride anions through electrostatic interaction by inducing H+, resulting in most lactic acid also being attracted. Hence, new hydrogen bonds were formed between choline chloride and protein, disrupting

In summary, these investigations delved into the interactions between choline chloride and lactic acid, as well as polyols, using FTIR, rheometer, and molecular modeling. It was found that the primary interaction among the components of prepared DESs was the formation of hydrogen bonds between the chlorine atom of choline chloride and the hydrogen atoms from the hydroxyl and carboxylic groups of lactic acid and polyols. Notably, the strength of this interaction increased with the growing number of hydroxyl groups in the polyols. However, the viscosity also showed an increasing trend with the number of hydroxyl groups in polyols, impacting the efficiency of chitin extraction. In this study, CCLaGly DES demonstrated superior extractive ability in obtaining pure chitin from LS compared to other DESs. Furthermore, the purity, crystallinity, and molecular weight of CCLaGly-Chitin were comparable to those of Chem-Chitin, indicating the promising application potential of CCLaGly DES in chitin extraction processes.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

As used herein, the term “substantially” means to a great or significant extent, but not completely.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. For example, a range of 0.1-2.0 includes 0.1, 0.2, 0.3, 0.4, 0.5, 0.6, 0.7, 0.8, 0.9, 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, “optional” or “optionally” means that the subsequently described event or circumstance may or may not occur, and that the description includes instances where said event or circumstance occurs and instances where it does not.

As used herein, a “ternary deep eutectic solvent (TDES)” refers to a solvent system useful for recovery of a target biomolecule from a biomass formed by mixing three components-typically a hydrogen bond acceptor (HBA), a first hydrogen bond donor (HBD1), and a second hydrogen bond donor (HBD2) in a HBA:HBD1:HBD2 molar ratio that can be adjusted to control a molecular weight, a viscosity, and/or a morphology of a target biomolecule to be recovered from the biomass. TDES systems are characterized by their ability to dissolve a wide variety of solutes, making them useful in applications such as extraction, synthesis, and formulation in various fields, including pharmaceuticals, food science, and chemical engineering.

As used herein, a “hydrogen bond acceptor” is a substance capable of accepting hydrogen bonds. In the context of a ternary deep eutectic solvent (TDES) system, “hydrogen bond acceptor” refers to a chemical species that possesses one or more electronegative atoms, such as nitrogen (N), oxygen (O), or sulfur(S), with available lone pairs of electrons. These lone pairs enable the hydrogen bond acceptor to interact with hydrogen atoms that are covalently bonded to other electronegative atoms, forming hydrogen bonds. In a TDES system, the hydrogen bond acceptor works in conjunction with a hydrogen bond donor to create a solvent environment characterized by a significantly reduced melting point compared to the individual components. The ability of the hydrogen bond acceptor to stabilize these interactions influences the unique physicochemical properties of the TDES, such as enhanced solubility and improved stability of solutes, which are essential for various applications in extraction, synthesis, and formulation. Examples of well-known hydrogen bond acceptors include choline chloride, trimethylammonium chloride, betaine, ammonium salts, phosphonium salts, glycolipids, phospholipids, carboxylic acids, and alcohols.

As used herein, a “hydrogen bond donor” is a substance that can donate hydrogen bonds. hydrogen bond acceptor is a substance capable of accepting hydrogen bonds. In the context of a ternary deep eutectic solvent (TDES) system, “hydrogen bond donor” refers to a molecular species that contains a hydrogen atom covalently bonded to an electronegative atom, such as nitrogen (N), oxygen (O), or sulfur(S). This electronegative atom attracts the shared electron density from the hydrogen atom, creating a partial positive charge on the hydrogen, which enables it to participate in hydrogen bonding interactions. In a TDES, the hydrogen bond donor interacts with a hydrogen bond acceptor, facilitating the formation of hydrogen bonds that contribute to the unique properties of the solvent system, including its reduced melting point and enhanced solvation characteristics. The ability of hydrogen bond donors to provide hydrogen atoms for bonding is essential for establishing the structural framework of TDES, influencing their viscosity, stability, and capacity to dissolve various solutes, thereby making them valuable for applications in extraction, synthesis, and various chemical processes.

The TDES system of the present disclosure contemplates using a first hydrogen bond donor (HBD1) comprising an organic acid and a second hydrogen bond donor (HBD2) comprising a polyol.

As used herein, an “organic acid” refers to a carbon-containing compound that possesses one or more carboxyl functional groups (—COOH) capable of donating protons (H+) in an aqueous environment. Organic acids are characterized by their ability to dissociate in solution, releasing hydrogen ions and contributing to the acidity of the solvent mixture. Examples of well-known organic acids suitable for use as hydrogen bond donors in TDES systems include acetic acid, citric acid, lactic acid, formic acid, benzoic acid, propionic acid, butyric acid, fumaric acid, tartaric acid, malic acid, oxalic acid, ascorbic acid, succinic acid, and glycolic acid.

As used herein, a “polyol” refers to a class of organic compounds that contain multiple hydroxyl (—OH) functional groups. These compounds are characterized by their ability to act as hydrogen bond donors due to the presence of these hydroxyl groups, which can participate in hydrogen bonding interactions with hydrogen bond acceptors in the TDES formulation. Polyols contribute to the unique properties of TDES, such as enhanced solubility, viscosity, and thermal stability, making them valuable components in various applications, including extraction, synthesis, and formulation processes. Examples of well-known polyols suitable for use in TDES systems include glycerol, sorbitol, mannitol, xylitol, erythritol, maltitol, arabitol, ribitol, and pentaerythritol.

As used herein, “control” refers to the ability to adjust the molar ratios of hydrogen bond acceptors (HBAs) and hydrogen bond donors (HBDs) within the solvent formulation to optimize specific features and properties of biomolecules recovered from biomass. This adjustment allows for the fine-tuning of the solvation environment, influencing critical parameters such as extraction efficiency, selectivity, stability, and functional characteristics of the biomolecules. By manipulating the molar ratios, one can achieve desired outcomes such as enhanced solubility of target compounds, improved recovery rates, and tailored interactions between the solvent and the biomolecules, thus facilitating the effective utilization of the TDES system in various extraction and processing applications.

As used herein, “molecular weight” refers to the mass of a single molecule, for example, of a biomolecule extracted from biomass, expressed in daltons (Da) or grams per mole (g/mol). The molecular weight of the biomolecule can be influenced by the extraction process, including the selection of solvent components, their molar ratios, and the conditions under which extraction occurs (such as temperature and time). By controlling these factors within the TDES systems of the present disclosure, it is possible to selectively extract biomolecules of desired molecular weights, which can impact their functional properties, bioactivity, and suitability for specific applications in fields such as pharmaceuticals, food science, and biochemistry.

As used herein, “viscosity” refers to the measure of a fluid's resistance to flow, which is influenced by the interactions between the solvent components and the solutes, including biomolecules extracted from biomass. Viscosity is typically expressed in centipoise (cP) or millipascal-seconds (mPa·s) and is a key property that affects the efficiency of extraction processes. In a TDES system of the present disclosure, the viscosity can be controlled through the selection and molar ratios of hydrogen bond donors (HBDs) and hydrogen bond acceptor (HBA). Controlling viscosity impacts the mass transfer and diffusion rates of biomolecules within the solvent, ultimately influencing the extraction efficiency, selectivity, and molecular weight of the biomolecules.

As used herein, “morphology” refers to the structural characteristics and physical form of the biomolecules extracted from biomass, including their shape, size, and arrangement at the microscopic or macroscopic level. Morphology can influence the functional properties, bioactivity, and stability of the extracted biomolecules. In a TDES system of the present disclosure, the morphology of the biomolecules can be affected by factors such as the molar ratios of hydrogen bond donors (HBDs) and hydrogen bond acceptor (HBA), extraction conditions (e.g., temperature, time, and concentration), and the viscosity of the solvent. By manipulating these parameters, one can control the molecular weight and the resulting morphology of the biomolecules, which may include variations in particle size, aggregation state, and structural integrity.

As used herein, “extracting” in the context of obtaining a target biomolecule from biomass using a ternary deep eutectic solvent (TDES) system of the present disclosure refers to selectively isolating and recovering specific biomolecules from complex biological materials. This process involves the dissolution of the target biomolecule into the TDES, which facilitates the release of the biomolecule from the biomass matrix by disrupting cellular structures and solubilizing the desired compounds. During the extraction process, the unique physicochemical properties of the TDES-such as its ability to form hydrogen bonds, low viscosity, and tunable solvation characteristics-enable effective interaction with the target biomolecule, enhancing its solubility and stability. The extraction may involve various operational parameters, including temperature, time, and concentration of the TDES, which can be optimized to maximize the yield and purity of the target biomolecule while minimizing the co-extraction of unwanted components. Overall, the term “extracting” encompasses the entire sequence of steps that lead to the efficient recovery of the target biomolecule from biomass for subsequent analysis or application.

As used herein, “contacting” refers to the process of bringing the TDES into physical proximity and interaction with a biomass containing target biomolecules of interest. This process involves the direct exposure of the biomass to the TDES, facilitating the transfer of the target biomolecule from the biomass matrix into the solvent phase. During the contacting process, the TDES penetrates the cellular structures of the biomass, disrupting them and solubilizing the desired biomolecules through interactions such as hydrogen bonding, van der Waals forces, and other molecular interactions. The effectiveness of the contacting step is influenced by various factors, including the duration of contact, the temperature of the system, the concentration of the TDES, and the physical state of the biomass (e.g., fresh, dried, or powdered). The goal of the contacting process is to maximize the extraction efficiency by ensuring that the TDES effectively interacts with and solubilizes the target biomolecule, thereby enhancing the overall yield and purity during the extraction process.

As used herein, “biomass” refers to any biological material derived from living organisms, including plant, animal, or microbial sources, that contains valuable biomolecules of interest. Biomass can encompass a wide range of materials, such as agricultural residues, food waste, algae, fungi, and animal by-products, which serve as natural reservoirs for proteins, carbohydrates, lipids, vitamins, and bioactive compounds. In the extraction process using a TDES system, biomass serves as the starting material from which target biomolecules are obtained. The unique physicochemical properties of the TDES facilitate the dissolution and recovery of specific biomolecules by disrupting the cellular structures within the biomass, thereby releasing the desired compounds into the solvent phase. The selection of biomass type and its preparation (e.g., drying, grinding, or extraction) are factors that can impact the efficiency and yield of the extraction process, ultimately influencing the quality and functionality of the extracted biomolecules.

As used herein, “target biomolecule” refers to a compound or group of compounds that is intended to be isolated and recovered from biomass during the extraction process. Target biomolecules can include a variety of substances such as proteins, peptides, polysaccharides, flavonoids, phenolic compounds, lipids, vitamins, and other bioactive compounds that are of interest for their functional, nutritional, or therapeutic properties. The successful extraction of the target biomolecule is influenced by factors such as the structure, polarity, and molecular weight of the biomolecule, as well as the specific interactions between the TDES and the biomolecule.

As used herein, “solubilize” refers to the process of converting a solid or insoluble compound within the biomass into a dissolved form within the TDES. This involves the interaction between the target biomolecule and the components of the TDES, which facilitates the breaking of intermolecular bonds within the biomolecule and the biomass matrix, allowing the biomolecule to enter the solvent phase. During the extraction process, the unique properties of the TDES such as its ability to form hydrogen bonds, low viscosity, and tunable polarity-enable effective solubilization of the target biomolecule. Solubilization is a step in the extraction process that affects the efficiency and yield of the target biomolecule, ultimately influencing its recovery.

As used herein, “separating” refers to the process of isolating and removing the solubilized target biomolecule from the residual biomass and any undissolved materials after the extraction process. This step typically involves techniques such as filtration, centrifugation, or sedimentation, which facilitate the physical separation of the dissolved biomolecule within the TDES from the solid components of the biomass that remain after solubilization. The objective of separating is to obtain a purified solution of the target biomolecule for further analysis, processing, or application, while minimizing contamination from residual biomass and other impurities.

As used herein, “residual biomass” refers to the remaining solid materials left after the extraction process, which may include cell walls, unextracted cellular components, and other non-solubilized plant, animal, or microbial tissues. Residual biomass may contain various materials that were not dissolved by the TDES and can still include complex mixtures of proteins, carbohydrates, lipids, and other biomolecules that were not targeted for extraction. The presence of residual biomass in the extraction system is a key consideration, as its effective separation is essential for achieving a high purity of the target biomolecule and ensuring the overall efficiency of the extraction process.

As used herein, “recovering” refers to the process of concentrating, purifying, and obtaining the solubilized target biomolecule after it has been separated from the residual biomass. This step typically involves methods such as evaporation, distillation, precipitation, or crystallization, which facilitate the removal of the TDES and any other solvents or impurities, resulting in the isolation of the biomolecule in a desirable form for subsequent use or analysis. The recovery process aims to maximize the yield and purity of the target biomolecule, ensuring that it retains its functional properties, stability, and bioactivity. The effectiveness of the recovery process is influenced by the properties of the biomolecule, the choice of recovery method, and the conditions employed during the extraction and separation steps.

As used herein, “seafood waste” refers to the by-products generated during the processing of seafood, which may include remnants such as fish heads, bones, shells, skin, and other non-edible parts that remain after the desirable portions of the seafood have been harvested. This waste is often rich in valuable biomolecules, including proteins, lipids, vitamins, and bioactive compounds, which can be extracted and utilized for various applications, including food ingredients, nutritional supplements, and pharmaceutical products. Seafood waste serves as a source material from which target biomolecules can be solubilized and extracted. The unique solvation properties of the TDES facilitate the disruption of the cellular and structural matrix of the seafood waste, promoting the release of the target biomolecules into the solvent. The efficient extraction of valuable components from seafood waste not only adds value to the by-products but also contributes to sustainable practices by minimizing waste and promoting the utilization of all parts of the seafood.

As used herein, “seaweed” refers to various types of marine macroalgae that grow in oceanic environments and are characterized by their photosynthetic capability. Seaweed includes species such as red algae (Rhodophyta), green algae (Chlorophyta), and brown algae (Phaeophyceae). Seaweed serves as a biomass source rich in bioactive compounds, including polysaccharides, proteins, vitamins, and minerals, which can be extracted using a TDES system of the present disclosure.

As used herein, “plant biomass” refers to the organic material derived from various parts of plants, including leaves, stems, roots, flowers, and seeds. This biomass is a renewable resource that contains a diverse array of biomolecules, such as carbohydrates, proteins, lipids, and phytochemicals. Plant biomass can be utilized as a feedstock for the extraction of target biomolecules, with a TDES system of the present disclosure facilitating the solubilization and recovery of these compounds.

As used herein, “agricultural waste” refers to the by-products generated during the cultivation, harvesting, and processing of agricultural crops. This includes materials such as crop residues (e.g., straw, husks, and leaves), fruit and vegetable peels, and other organic by-products that are typically discarded or underutilized. Agricultural waste can be a valuable source of biomolecules, including fibers, antioxidants, and essential oils, which can be extract using a TDES system of the present disclosure to promote sustainability and resource efficiency in agricultural practices.

As used herein, “lignocellulosic biomass” refers to plant-based materials that are primarily composed of cellulose, hemicellulose, and lignin. This type of biomass includes wood, straw, agricultural residues, and other plant materials that have a complex structure.

Lignocellulosic biomass has the potential to yield a variety of bioactive compounds, such as fermentable sugars, phenolic compounds, and other phytochemicals.

As used herein, “magnetic stirring” refers to a method of mixing liquids or slurries by using a rotating magnetic field to drive a magnetic stir bar (or stirring magnet) placed within the liquid. This technique provides efficient and uniform mixing, which is particularly useful when treating biomass to ensure homogeneity in the solvent and biomass mixture during extraction processes. In the context of treating seafood waste to extract a biomolecule, magnetic stirring can be performed by placing the biomass and a ternary deep eutectic solvent (TDES) in a container equipped with a magnetic stirrer. The stirrer is activated, causing the stir bar to rotate and mix the biomass with the TDES, facilitating the release of target biomolecules into the solvent. In a laboratory setting, seafood waste can be mixed with TDES in a beaker placed on a magnetic stirrer, allowing the stir bar to continuously agitate the mixture for a predetermined period, enhancing solubilization and extraction of target biomolecules. In a larger-scale application, a reactor vessel containing the seafood waste and TDES can be equipped with an internal magnetic stirrer to maintain constant mixing during the extraction process, ensuring optimal interaction between the solvent and biomass.

As used herein, “mass ratio” refers to the proportion of the mass of one component in a mixture compared to the mass of another component. In the context of TDES systems for biomass extraction, mass ratio is useful for optimizing the solvent formulation. For example, the mass ratio of a first hydrogen bond donor and a second hydrogen bond donor to hydrogen bond acceptor can be adjusted to achieve desired extraction efficiencies.

As used herein, “extract” refers to the solution obtained after solubilizing target biomolecules from biomass using a solvent, such as a TDES. The extract contains the desired biomolecules and may also include other soluble compounds, depending on the extraction conditions and the composition of the biomass.

As used herein, “precipitate” refers to the solid material that forms and separates from a solution during a chemical reaction or physical process, such as solvent evaporation or cooling. In the context of biomolecule extraction, a precipitate may form when certain components in the extract are no longer soluble under specific conditions, allowing for the recovery of biomolecules through filtration or centrifugation.

As used herein, “drying” refers to the process of removing moisture from a substance, typically by applying heat or reducing pressure. In the context of extracting biomolecules from seafood waste using a TDES, drying may be employed to concentrate the extract or to prepare the precipitate for storage or further processing. Common drying methods include air drying, freeze drying, and spray drying.

As used herein, “recycling a TDES” refers to the process of reusing a ternary deep eutectic solvent after it has been used in an extraction process. This typically involves separating the solvent from the extracted biomass and any residual biomolecules, followed by purification or reconstitution to restore the solvent's properties for subsequent extractions. Recycling is advantageous for sustainability and cost-effectiveness in extraction processes. The TDES systems of the present disclosure can be reused for at least one cycle, at least two cycle, at least three cycles, at least four cycles, or at least five or more cycles.

As used herein, “enzymatic pretreatment” refers to the application of enzymes to biomass prior to extraction, aimed at enhancing the release of target biomolecules. This process can help break down complex structures in the biomass, making it easier for the solvent to solubilize the desired compounds. Though the TDES systems of the present disclosure are capable of producing high yields of target biomolecules with controllable molecular weight, viscosity, and morphology, without comprising purity and without enzymes, enzymes can optionally be used in a pretreatment step. Exemplary enzymes include, without limitation, proteases, cellulases, pectinases, laccases.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include 11C, 13C, and 14C. Accordingly, the compounds disclosed herein may include heavy or radioactive isotopes in the structure of the compounds or as substituents attached thereto. Examples of useful heavy or radioactive isotopes include 18F, 15N, 180, 76Br, 1251 and 131T.

A significant change is any detectable change that is statistically significant in a standard parametric test of statistical significance such as Student's T-test, where p<0.05.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

For reasons of completeness, various aspects of the disclosure are set out in the following numbered clauses:

Clause 1. A ternary deep eutectic solvent (TDES) system for recovery of a target biomolecule from a biomass, the system comprising:
(a) a hydrogen bond acceptor (HBA) comprising betaine or choline chloride;
(b) a first hydrogen bond donor (HBD1) comprising an organic acid; and
(c) a second hydrogen bond donor (HBD2) comprising a polyol,
wherein the system comprises a HBA:HBD1:HBD2 molar ratio that can be adjusted to control a molecular weight, a viscosity, and/or a morphology of the target biomolecule recovered from the biomass.

Clause 2. The system of clause 1, wherein the organic acid is ascorbic acid, citric acid, lactic acid, malic acid, or any combination thereof.

Clause 3. The system of clause 1, wherein the polyol is ethylene glycol, glycerol, pentaerythritol, sorbitol, xylitol, or any combination thereof.

Clause 4. The system of clause 1, wherein the HBA:HBD1:HBD2 molar ratio can be adjusted from about 1:0.05:0.1 to about 2:2:1.5 to control the molecular weight, the viscosity, and/or the morphology of the target biomolecule recovered from the biomass.

Clause 5. The system of clause 4, wherein the HBA is choline chloride, the HBD1 is lactic acid, and the HBD2 is glycerol in a molar ratio of from about 1:1.9:0.1 to about 1:0.7:1.3.

Clause 6. The system of clause 5, wherein the target biomolecule comprises chitin, the biomass comprises seafood waste, and the molecular weight of the chitin recovered ranges from about 200 kDa to about 550 kDa without comprising purity and/or yield.

Clause 7. The system of clause 4, wherein the HBA is choline chloride, the HBD1 is malic acid, and the HBD2 is glycerol in a molar ratio of from about 2:1.5:0.5 to about 2:1:1.

Clause 8. The system of clause 7, wherein the target biomolecule comprises chitin, the biomass comprises seafood waste, and the molecular weight of the chitin recovered ranges from about 200 kDa to about 550 kDa without comprising purity and/or yield.

Clause 9. The system of clause 1, wherein the TDES system:
(i) comprises lactic acid and has a viscosity of from about 15 mPa·s to about 25 mPa·s at 80° C.; or
(ii) comprises malic acid and has a viscosity of from about 80 mPa·s to about 250 mPa·s at 80° C.

Clause 10. A process for extracting a target biomolecule from a biomass, the process comprising:
(a) contacting a biomass comprising a target biomolecule with a TDES system according to clause 1 under conditions effective to solubilize the target biomolecule;
(b) separating the TDES containing the solubilized biomolecule from a residual biomass; and
(c) recovering the target biomolecule from the TDES.

Clause 11. The process of clause 10, wherein the biomass comprises seafood waste, seaweed, plant biomass, agricultural waste, lignocellulosic biomass, or any combination thereof.

Clause 12. The process of clause 10, wherein the target biomolecule is a protein, an enzyme, a polysaccharide, lipid or any combination thereof.

Clause 13. The process of clause 10, wherein the biomass is seafood waste comprising shrimp shells, lobster shells, crab shells, fish scales or any combination thereof, and the target biomolecule is chitin, collagen or a combination thereof.

Clause 14. The process of clause 10, wherein the contacting step (a) is performed at a temperature of about 50° C. to about 90° C.

Clause 15. The process of clause 10, further comprising the step of adjusting the pH of the TDES system to precipitate the target biomolecule after separation in step (b).

Clause 16. A process for treating a biomass comprising seafood waste, the process comprising:
(a) magnetically stirring the biomass with a TDES according to clause 1 at a mass ratio of from about 0.5:10 to about 1.5:30 for a period of from about 1 hours to about 3 hours at a temperature ranging from about 50° C. to about 90° C. to produce an extract comprising chitin;
(b) centrifuging the extract for a period ranging from about 5 minutes to about 15 minutes to produce a supernatant comprising a precipitate of the extract; and
(c) drying the supernatant at a temperature ranging from about 35° C. to about 45° C. for a period of from about 16 hours to about 32 hours to produce a powder comprising chitin.

Clause 17. The process of clause 16, further comprising cooling the extract produced in step (a) to ambient temperature prior to centrifuging the extract in step (b) and/or neutralizing the pH of the supernatant produced in step (b) prior to drying the supernatant in step (c).

Clause 18. The process of clause 16, further comprising the step of recycling the TDES for use in subsequent extractions.

Clause 19. The process of clause 16, further comprising optionally performing an enzymatic treatment step prior to step (a) to enhance the extraction efficiency of the TDES.

Clause 20. A process for preparing a TDES system for recovery of a target biomolecule from a biomass, comprising mixing a HBA comprising choline chloride, a HBD1 comprising an organic acid; and a HBD2 comprising a polyol at a HBA:HBD1:HBD2 molar ratio selected based on a desired molecular weight, viscosity, and/or morphology of a target biomolecule to be recovered from a biomass, wherein the mixing is performed at a temperature ranging from about 75° C. to about 95° C. for a time period ranging from about 1 hour to about 3 hours until a homogenous liquid comprising the TDES system is produced.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A ternary deep eutectic solvent (TDES) system for recovery of a target biomolecule from a biomass, the system comprising:
(a) a hydrogen bond acceptor (HBA) comprising betaine or choline chloride;
(b) a first hydrogen bond donor (HBD1) comprising an organic acid; and
(c) a second hydrogen bond donor (HBD2) comprising a polyol,
wherein the system comprises a HBA:HBD1:HBD2 molar ratio that can be adjusted to control a molecular weight, a viscosity, and/or a morphology of the target biomolecule recovered from the biomass.

2. The system of claim 1, wherein the organic acid is ascorbic acid, citric acid, lactic acid, malic acid, or any combination thereof.

3. The system of claim 1, wherein the polyol is ethylene glycol, glycerol, pentaerythritol, sorbitol, xylitol, or any combination thereof.

4. The system of claim 1, wherein the HBA:HBD1:HBD2 molar ratio can be adjusted from about 1:0.05:0.1 to about 2:2:1.5 to control the molecular weight, the viscosity, and/or the morphology of the target biomolecule recovered from the biomass.

5. The system of claim 4, wherein the HBA is choline chloride, the HBD1 is lactic acid, and the HBD2 is glycerol in a molar ratio of from about 1:1.9:0.1 to about 1:0.7:1.3.

6. The system of claim 5, wherein the target biomolecule comprises chitin, the biomass comprises seafood waste, and the molecular weight of the chitin recovered ranges from about 200 kDa to about 550 kDa without comprising purity and/or yield.

7. The system of claim 4, wherein the HBA is choline chloride, the HBD1 is malic acid, and the HBD2 is glycerol in a molar ratio of from about 2:1.5:0.5 to about 2:1:1.

8. The system of claim 7, wherein the target biomolecule comprises chitin, the biomass comprises seafood waste, and the molecular weight of the chitin recovered ranges from about 200 kDa to about 550 kDa without comprising purity and/or yield.

9. The system of claim 1, wherein the TDES system:
(i) comprises lactic acid and has a viscosity of from about 15 mPa·s to about 25 mPa·s at about 80° C.; or
(ii) comprises malic acid and has a viscosity of from about 80 mPa·s to about 250 mPa·s at about 80° C.

10. A process for extracting a target biomolecule from a biomass, the process comprising:
(a) contacting a biomass comprising a target biomolecule with a TDES system according to claim 1 under conditions effective to solubilize the target biomolecule;
(b) separating the TDES containing the solubilized biomolecule from a residual biomass; and
(c) recovering the target biomolecule from the TDES.

11. The process of claim 10, wherein the biomass comprises seafood waste, seaweed, plant biomass, agricultural waste, lignocellulosic biomass, or any combination thereof.

12. The process of claim 10, wherein the target biomolecule is a protein, an enzyme, a polysaccharide, lipid or any combination thereof.

13. The process of claim 10, wherein the biomass is seafood waste comprising shrimp shells, lobster shells, crab shells, fish scales or any combination thereof, and the target biomolecule is chitin, collagen or a combination thereof.

14. The process of claim 10, wherein the contacting step (a) is performed at a temperature of about 50° C. to about 90° C.

15. The process of claim 10, further comprising the step of adjusting the pH of the TDES system to precipitate the target biomolecule after separation in step (b).

16. A process for treating a biomass comprising seafood waste, the process comprising:
(a) magnetically stirring the biomass with a TDES according to claim 1 at a mass ratio of from about 0.5:10 to about 1.5:30 for a period of from about 1 hours to about 3 hours at a temperature ranging from about 50° C. to about 90° C. to produce an extract comprising chitin;
(b) centrifuging the extract for a period ranging from about 5 minutes to about 15 minutes to produce a supernatant comprising a precipitate of the extract; and
(c) drying the supernatant at a temperature ranging from about 35° C. to about 45° C. for a period of from about 16 hours to about 32 hours to produce a powder comprising chitin.

17. The process of claim 16, further comprising cooling the extract produced in step (a) to ambient temperature prior to centrifuging the extract in step (b) and/or neutralizing the pH of the supernatant produced in step (b) prior to drying the supernatant in step (c).

18. The process of claim 16, further comprising the step of recycling the TDES for use in subsequent extractions.

19. The process of claim 16, further comprising optionally performing an enzymatic treatment step prior to step (a) to enhance the extraction efficiency of the TDES.

20. A process for preparing a TDES system for recovery of a target biomolecule from a biomass, comprising mixing a HBA comprising betaine or choline chloride, a HBD1 comprising an organic acid; and a HBD2 comprising a polyol at a HBA:HBD1:HBD2 molar ratio selected based on a desired molecular weight, viscosity, and/or morphology of a target biomolecule to be recovered from a biomass, wherein the mixing is performed at a temperature ranging from about 75° C. to about 95° C. for a time period ranging from about 1 hour to about 3 hours until a homogenous liquid comprising the TDES system is produced.
